SEQUENCE LISTING (72 sequences)

SEQ ID NO: 1 - DNA, 24 bp, Artificial Sequence
  Description of Artificial Sequence Synthetic oligonucleotide
ctaaacaaacaattccaaacatag

SEQ ID NO: 2 - DNA, 24 bp, Artificial Sequence
  Description of Artificial Sequence Synthetic oligonucleotide
aaaagacatagaaaatacgatagt

SEQ ID NO: 3 - DNA, 1248 bp, Drosophila melanogaster
aagtgttactatctaaacacatttcaaacaattcttaacaaacaattccaaacatacaattccacttaccacttaccgaccaaattacgagtttacaatggacaaagctgaacgcgactactggcatcttcgatccttggaaatcgaagaggagccgcgatttccgccaacaaacgtcgctgatccactaaccgcacgcaatctgttccagctctacgtcaacaccttcattggagccaatctggccgagtcgtgtgttttcccattggacgtggccaagacccggatgcaggtagatggcgagcaggccaagaagacgggtaaagcgatgccaactttccgtgcaactcttaccaacatgatccgagtggagggattcaagtcgctctacgccggcttctcggcaatggtgacccgaaactttatcttcaactcgttacgtgttgttctctacgacgttttccggcgcccttttctctaccagaacgaacggaacgaggaagtgctcaagatctacatggcgctgggatgcagcttcaccgcaggctgcattgcccaggcactggccaatccctttgacatcgtcaaggtgcgaatgcagacggaaggacgccgccgccagctgggctatgatgtgcgggtgaacagcatggtgcaggccttcgtggacatctaccgccgtggcggactgcccagtatgtggaagggtgtagggcccagctgcatgcgtgcctgcctgatgacgaccggcgatgtgggcagttacgatatcagtaagcgcaccttcaagcgcctgctggacttggaggaaggcctgccactgcgtttcgtgtcttccatgtgcgccggactaacggcatccgtgctcagcacgccggcgaacgtgatcaagtcgcggatgatgaaccagccggtgaacgagagcggcaagaatctgtactacaagaactccctcgactgcattaggaagctggtcagggaggagggtgtcctcacgttgtataagggcctcatgcccacttggtttcgcctgggaccgttctcagtgctcttttggctgtccgtcgagcagctgcgtcagtggaaaggccagagtggattttaggagcaaactatcaatcttactatcgtattttgtatgtcttttaacacgcaataaaaagggtgcaagtcaaaccatctattatacatattataaatataactttaatcccaaaaaaaaaaaaaaaaactcgtgccgaattcgat

SEQ ID NO: 4 - DNA, 1008 bp, Drosophila melanogaster
  CDS (1)..(1005)
atggacaaagctgaacgcgactactggcatcttcgatccttggaaatcgaagaggagccgcgatttccgccaacaaacgtcgctgatccactaaccgcacgcaatctgttccagctctacgtcaacaccttcattggagccaatctggccgagtcgtgtgttttcccattggacgtggccaagacccggatgcaggtagatggcgagcaggccaagaagacgggtaaagcgatgccaactttccgtgcaactcttaccaacatgatccgagtggagggattcaagtcgctctacgccggcttctcggcaatggtgacccgaaactttatcttcaactcgttacgtgttgttctctacgacgttttccggcgcccttttctctaccagaacgaacggaacgaggaagtgctcaagatctacatggcgctgggatgcagcttcaccgcaggctgcattgcccaggcactggccaatccctttgacatcgtcaaggtgcgaatgcagacggaaggacgccgccgccagctgggctatgatgtgcgggtgaacagcatggtgcaggccttcgtggacatctaccgccgtggcggactgcccagtatgtggaagggtgtagggcccagctgcatgcgtgcctgcctgatgacgaccggcgatgtgggcagttacgatatcagtaagcgcaccttcaagcgcctgctggacttggaggaaggcctgccactgcgtttcgtgtcttccatgtgcgccggactaacggcatccgtgctcagcacgccggcgaacgtgatcaagtcgcggatgatgaaccagccggtgaacgagagcggcaagaatctgtactacaagaactccctcgactgcattaggaagctggtcagggaggagggtgtcctcacgttgtataagggcctcatgcccacttggtttcgcctgggaccgttctcagtgctcttttggctgtccgtcgagcagctgcgtcagtggaaaggccagagtggattttag

SEQ ID NO: 5 - protein, 335 aa, Drosophila melanogaster
MDKAERDYWHLRSLEIEEEPRFPPTNVADPLTARNLFQLYVNTFIGANLAESCVFPLDVAKTRMQVDGEQAKKTGKAMPTFRATLTNMIRVEGFKSLYAGFSAMVTRNFIFNSLRVVLYDVFRRPFLYQNERNEEVLKIYMALGCSFTAGCIAQALANPFDIVKVRMQTEGRRRQLGYDVRVNSMVQAFVDIYRRGGLPSMWKGVGPSCMRACLMTTGDVGSYDISKRTFKRLLDLEEGLPLRFVSSMCAGLTASVLSTPANVIKSRMMNQPVNESGKNLYYKNSLDCIRKLVREEGVLTLYKGLMPTWFRLGPFSVLFWLSVEQLRQWKGQSGF

SEQ ID NO: 6 - DNA, 2953 bp, Drosophila melanogaster
ctgctgggaattcggcacgagaagtcgattagtcgtttttcgttggagagcttgctgttcgcatttatccgcctgctcgcgcgtagaaaagttgttacttaatgttcattgctgccacacgtgcttgattattaattgcagtgcgctaatcagtcgcttgtcagctgagcagaccacaaaaaacgaaggcaattatttaggctgccctttcagttcggcattacataagaaacgaatggccaaacgaaaataattttgaaacaatttcggagttaatattgcatcagcaattctgaacagcagatacacaaaaagggcagcaatttggccaacaaagtcttcataaacgacaacaaacgagtgaaaatatgagctgggcaacaacaaagcaaaggcacaaatgtttcaactgtagcagcggcagcaacaaaaacaaccggagcagcggcagcagcagcaacaacaacaactgtgcgcctagcaacaagataatttcgacgagcgatttcctttgatttgcgcccatctgtgtcgaaagcagccgaaaagccaaagagtgtaaatagcacgtagttttagtacaattcactgtagcaaaagtgagtttcaaccggggcagccaataagcctgcgatagcaacaataatacagaacgagcgcaaaagaaatcaccagagattgcacaatatgaatccgatcaaggcacagtcaacgggcagtcccaagaaattcaacgtattcgcacacgtcaagtacgagcatttggttgccggagtatccggcggagtggtgtccacactcattctacatcccctggatttgatcaagattcgattcgcagttaacgatggccggacagctacggtgccgcaataccggggactgagcagcgccttcaccacgattttccggcaagagggcttccgcggactctacaaaggcgtcacccccaatgtctggggatcgggctcctcttggggcctgtacttcatgttctacaacaccattaagacatttatccaaggaggaaacacgaccatgccattgggccccacaatgaacatgcttgcagctgctgagtcgggaattctcaccctgctgctgaccaaccccatctgggtggtgaagacgcgtctctgcctgcagtgcgatgccgcgagtagtgccgagtacaggggcatgatccacgccttgggccagatatacaaggaggagggaatccgtggcctgtaccgcggctttgttcccggcatgttgggcgtctcccacggagccatccagttcatgacctacgaggagctgaagaacgcctacaacgaatatcgcaaactgcccatcgacacgaagctggccaccaccgagtacttggccttcgcggctgtctccaagctgatcgcagcggcggccacctacccgtaccaggtggtccgggcacggctgcaggaccaccatcaccgatacaacggcacctgggactgcatcaaacagacttggaggtacgagcgcatgcgaggtttctataagggcctggtgccctacctggtccacgtcacgcccaacatctgcatggtcatgctgatctgggagaagctgaccagctagatggagtattagtactagatcatcgaatctggaatctgacagagaatttaagctaagcacctagaatacacgaatctttctcgtttcctccgatgtgcagctaacagcagaaaatgacaaacttattctgtattattgttgtaactcgatttcggtttagccctagcaccttactttagccttaagtgtattccatatctagtttattgtacatccttcactcccaacttcgaaagtaactgatgtgatcggggcgtcgtcaaaatagcccctgcccatgaaatgtaatttcaaaaacgcatttcttgcactcttcatcaaaacaaacacatatttagttcttagtttagttaaattatttattctataacggcttggaattgtgtaagcacaaagaaaaaccagttactgtttagttgatacaaaatttcgttttggcaaaaagattcttcatagtttttgaaaatggtttataattttaaggataaaccttgacatttatgtaaataagcataaacgcattgtgaaaccaacttagtcctacagtggaacaatcttattgaaatcaaagaaaagataagaaatgcgaaattacttacccatattaaaaggccacgatggcagacctaaatgaaaaccaagaaacaaactaaagcaaacactgtcagcgcgtccagcctgcgtcttttcatttgtccaagggctcatggcagcttgagtaagtagtccttgctccttccgctccctaagtcttcgatcacccccttaattgtgcagaatctctttgctaatctgaaacagccaatgcgcccttactgtaagtagatgtagctttcagttctcagtctctaaccgcattgtgtttggttctctttgcaggtttgagggctacagaggcttctacaaggggctgaaggcgagtttaacccgagtagtgcctgcctgcatggtcacctttctggtgtacgagaacgtctcgcatttcctgctcgccaggcggaagcgaattgagactaaagaggatgcgtcggacgtgtgattttcctttggttggattccttttaggcttctaagatacatatatcccctcacgacatttccatatgtctctatagtcagcgggcagttgcaatcgtgctgagcatgcgcagctgcttttagtttaagtttagttagttgttgcgaagacttatttggtcttttcgtagtaagtaaagtgattgttgttcctaagtgtggatcagaatggatttgtgattgaattcagacgaaagtgttacaatatacctgtgaaatataagtccatagcctcactcttgcaaataaaccaaatctttgtgagcccaaaaaa

SEQ ID NO: 7 - DNA, 915 bp, Drosophila melanogaster
  CDS (1)..(912)
atgaatccgatcaaggcacagtcaacgggcagtcccaagaaattcaacgtattcgcacacgtcaagtacgagcatttggttgccggagtatccggcggagtggtgtccacactcattctacatcccctggatttgatcaagattcgattcgcagttaacgatggccggacagctacggtgccgcaataccggggactgagcagcgccttcaccacgattttccggcaagagggcttccgcggactctacaaaggcgtcacccccaatgtctggggatcgggctcctcttggggcctgtacttcatgttctacaacaccattaagacatttatccaaggaggaaacacgaccatgccattgggccccacaatgaacatgcttgcagctgctgagtcgggaattctcaccctgctgctgaccaaccccatctgggtggtgaagacgcgtctctgcctgcagtgcgatgccgcgagtagtgccgagtacaggggcatgatccacgccttgggccagatatacaaggaggagggaatccgtggcctgtaccgcggctttgttcccggcatgttgggcgtctcccacggagccatccagttcatgacctacgaggagctgaagaacgcctacaacgaatatcgcaaactgcccatcgacacgaagctggccaccaccgagtacttggccttcgcggctgtctccaagctgatcgcagcggcggccacctacccgtaccaggtggtccgggcacggctgcaggaccaccatcaccgatacaacggcacctgggactgcatcaaacagacttggaggtacgagcgcatgcgaggtttctataagggcctggtgccctacctggtccacgtcacgcccaacatctgcatggtcatgctgatctgggagaagctgaccagctag

SEQ ID NO: 8 - protein, 304 aa, Drosophila melanogaster
MNPIKAQSTGSPKKFNVFAHVKYEHLVAGVSGGVVSTLILHPLDLIKIRFAVNDGRTATVPQYRGLSSAFTTIFRQEGFRGLYKGVTPNVWGSGSSWGLYFMFYNTIKTFIQGGNTTMPLGPTMNMLAAAESGILTLLLTNPIWVVKTRLCLQCDAASSAEYRGMIHALGQIYKEEGIRGLYRGFVPGMLGVSHGAIQFMTYEELKNAYNEYRKLPIDTKLATTEYLAFAAVSKLIAAAATYPYQVVRARLQDHHHRYNGTWDCIKQTWRYERMRGFYKGLVPYLVHVTPNICMVMLIWEKLTS

SEQ ID NO: 9 - DNA, 948 bp, Homo sapiens
  CDS (1)..(945)
atgacgggccagggccagtcggcgtccgggtcgtcggcgtggagcacggtattccgccacgtccggtatgagaacctgatagcgggcgtgagcggcggcgtcttatccaaccttgcgctgcatccgctcgacctcgtgaagatccgcttcgccgtgagtgatggattggaactgagaccgaaatataatggaattttacattgcttgactaccatttggaaacttgatggactacggggactttatcaaggagtaaccccaaatatatggggtgcaggtttatcctggggactctactttttcttttacaatgccatcaagtcatataaaacagaaggaagagctgaacgtttagaggcaacagaataccttgtctcagctgctgaagctggagccatgaccctctgcattacaaacccattatgggtaacaaaaactcgccttatgttacagtatgatgctgttgttaactccccacacccgcaatataaaggaatgtttgatacacttgtgaaaatatataagtatgaaggtgtgcgtggattatataagggatttgttcctgggctgtttggaacatcgcacggtgcccttcagtttatggcatatgaattgctgaagttgaagtacaaccagcatatcaatagattaccagaagcccagttgagcacagtagaatatatatctgttgcagcactatccaaaatatttgctgtcgcagcaacatacccatatcaagtcgtaagagctcgtcttcaggatcaacacatgttttacagtggtgtaatagatgtaatcacaaagacatggaggaaagaaggcgtcggtggattttacaagggaattgctcctaatttgattagagtgactccagcctgctgtattacctttgtggtatatgaaaacgtctcacattttttacttgaccttagagaaaagagaaagtaa

SEQ ID NO: 10 - protein, 315 aa, Homo sapiens
MTGQGQSASGSSAWSTVFRHVRYENLIAGVSGGVLSNLALHPLDLVKIRFAVSDGLELRPKYNGILHCLTTIWKLDGLRGLYQGVTPNIWGAGLSWGLYFFFYNAIKSYKTEGRAERLEATEYLVSAAEAGAMTLCITNPLWVTKTRLMLQYDAVVNSPHPQYKGMFDTLVKIYKYEGVRGLYKGFVPGLFGTSHGALQFMAYELLKLKYNQHINRLPEAQLSTVEYISVAALSKIFAVAATYPYQVVRARLQDQHMFYSGVIDVITKTWRKEGVGGFYKGIAPNLIRVTPACCITFVVYENVSHFLLDLREKRK

SEQ ID NO: 11 - DNA, 951 bp, Mus musculus
  CDS (1)..(948)
atgacaggccagggccagtcggctgccgggtcggcggcgtggagcgtggtgttccgccacgtccggtacgagaacctggtggctggcgtgagtggcggggtcttgtccaacctggcgctgcacccgctcgacctcgtgaagatccgcttcgctgtgagtgatggactggaagtaagaccaaaatataaaggaattttgcattgcttggctaccatttggaaagttgatggactacgaggactttatcaaggagtaaccccgaatgtgtggggtgccggtttatcctggggactctactttttcttttacaatgccatcaaatcgtataagacagagggaagagctgaacagttagagccattagagtacctcgtctcagctgctgaagctggagccatgactctgtgcattacaaacccattatgggtgacgaaaactcgccttatgttacaatatggtggtgttgctagcccttcacagagacagtataaaggaatgtttgatgcacttgtgaaaatatataaatatgaaggtgtgcgtggattatacaagggatttgtccctgggctgtttggaacatcacatggtgcccttcagtttatggcatatgagttgctaaagttgaagtacaacaaacacatcaatagattaccggaagcccagctgagtacagcagaatacatctctgtcgcagcgctatccaaaatatttgccgtagcagcaacatacccgtatcaggttgtgagagcccgccttcaggatcagcatgtgtcttatggtggtgtaacagatgtgatcacaaagacgtggaggaaagaaggcatcggtggattttacaaaggaattgcccccaatctgattagagtgactccagcctgctgcatcacctttgtggtttatgaaaatgtctctcactttttatatgaccttagagaaaagaaagtgggctaa

SEQ ID NO: 12 - protein, 316 aa, Mus musculus
MTGQGQSAAGSAAWSVVFRHVRYENLVAGVSGGVLSNLALHPLDLVKIRFAVSDGLEVRPKYKGILHCLATIWKVDGLRGLYQGVTPNVWGAGLSWGLYFFFYNAIKSYKTEGRAEQLEPLEYLVSAAEAGAMTLCITNPLWVTKTRLMLQYGGVASPSQRQYKGMFDALVKIYKYEGVRGLYKGFVPGLFGTSHGALQFMAYELLKLKYNKHINRLPEAQLSTAEYISVAALSKIFAVAATYPYQVVRARLQDQHVSYGGVTDVITKTWRKEGIGGFYKGIAPNLIRVTPACCITFVVYENVSHFLYDLREKKVG

SEQ ID NO: 13 - DNA, 951 bp, Mus musculus
  CDS (1)..(948)
atgacaggccagggccagtcggctgccgggtcggcggcgtggagcgcggtgttccgccacgtccggtacgagaacctggtggctggcgtgagtggcggggtcttgtccaacctggcgctgcacccgctcgacctcgtgaagatccgcttcgctgtgagtgatggactggaagtaagaccaaaatataaaggaattttgcattgcttggctaccatttggaaagttgatggactacgaggactttatcaaggagtaaccccgaatgtgtggggtgccggtttatcctggggactctactttttcttttacaatgccatcaaatcgtataagacagagggaagagctgaacagttagagccattagagtacctcgtctcagctgctgaagctggagccatgactctgtgcattacaaacccattatgggtgacgaaaactcgccttatgttacaatatggtggtgttgctagcccttcacagagacagtataaaggaatgtttgatgcacttgtgaatatatataaatatgaaggtgtgcgtggattatacaagggatttgtccctgggctgtttggaacatcacatggtgcccttcagtttatggcatatgagttgctaaagttgaagtacaacaaacacatcaatagattaccggaagcccagctgagtacagcagaatacatctctgtcgcagcgctatccaaaatatttgccgtagcagcaacatacccgtatcaggttgtgagagcccgccttcaggatcagcatgtgtcttatggtggtgtaacagatgtgatcacaaagacgtggaggaaagaaggcatcggtggattttacaaaggaattgcccccaatctgattagggtgactccagcctgctgcatcacctttgtggtttatgaaaatgtctctcactttttatatgaccttagagaaaagaaagtgagctaa

SEQ ID NO: 14 - protein, 316 aa, Mus musculus
MTGQGQSAAGSAAWSAVFRHVRYENLVAGVSGGVLSNLALHPLDLVKIRFAVSDGLEVRPKYKGILHCLATIWKVDGLRGLYQGVTPNVWGAGLSWGLYFFFYNAIKSYKTEGRAEQLEPLEYLVSAAEAGAMTLCITNPLWVTKTRLMLQYGGVASPSQRQYKGMFDALVNIYKYEGVRGLYKGFVPGLFGTSHGALQFMAYELLKLKYNKHINRLPEAQLSTAEYISVAALSKIFAVAATYPYQVVRARLQDQHVSYGGVTDVITKTWRKEGIGGFYKGIAPNLIRVTPACCITFVVYENVSHFLYDLREKKVS

SEQ ID NO: 15 - protein, 16 aa, Drosophila melanogaster
LVAGVSGGVVSTLILH

SEQ ID NO: 16 - protein, 28 aa, Drosophila melanogaster
VKYEHLVAGVSGGVVSTLILHPLDLIKI

SEQ ID NO: 17 - protein, 23 aa, Drosophila melanogaster
GVTPNVWGSGSSWGLYFMFYNTI

SEQ ID NO: 18 - protein, 31 aa, Drosophila melanogaster
GLYKGVTPNVWGSGSSWGLYFMFYNTIKTFI

SEQ ID NO: 19 - protein, 23 aa, Drosophila melanogaster
MNMLAAAESGILTLLLTNPIWVV

SEQ ID NO: 20 - protein, 27 aa, Drosophila melanogaster
GPTMNMLAAAESGILTLLLTNPIWVVK

SEQ ID NO: 21 - protein, 23 aa, Drosophila melanogaster
GLYRGFVPGMLGVSHGAIQFMTY

SEQ ID NO: 22 - protein, 25 aa, Drosophila melanogaster
RGLYRGFVPGMLGVSHGAIQFMTYE

SEQ ID NO: 23 - protein, 21 aa, Drosophila melanogaster
EYLAFAAVSKLIAAAATYPYQ

SEQ ID NO: 24 - protein, 28 aa, Drosophila melanogaster
LATTEYLAFAAVSKLIAAAATYPYQVVR

SEQ ID NO: 25 - protein, 22 aa, Drosophila melanogaster
FYKGLVPYLVHVTPNICMVMLI

SEQ ID NO: 26 - protein, 24 aa, Drosophila melanogaster
GFYKGLVPYLVHVTPNICMVMLIW

SEQ ID NO: 27 - protein, 16 aa, Homo sapiens
LIAGVSGGVLSNLALH

SEQ ID NO: 28 - protein, 26 aa, Homo sapiens
YENLIAGVSGGVLSNLALHPLDLVKI

SEQ ID NO: 29 - protein, 24 aa, Homo sapiens
LYQGVTPNIWGAGLSWGLYFFFYN

SEQ ID NO: 30 - protein, 27 aa, Homo sapiens
GLYQGVTPNIWGAGLSWGLYFFFYNAI

SEQ ID NO: 31 - protein, 21 aa, Homo sapiens
YLVSAAEAGAMTLCITNPLWV

SEQ ID NO: 32 - protein, 23 aa, Homo sapiens
EYLVSAAEAGAMTLCITNPLWVT

SEQ ID NO: 33 - protein, 23 aa, Homo sapiens
GLYKGFVPGLFGTSHGALQFMAY

SEQ ID NO: 34 - protein, 27 aa, Homo sapiens
RGLYKGFVPGLFGTSHGALQFMAYELL

SEQ ID NO: 35 - protein, 22 aa, Homo sapiens
VEYISVAALSKIFAVAATYPYQ

SEQ ID NO: 36 - protein, 29 aa, Homo sapiens
QLSTVEYISVAALSKIFAVAATYPYQVVR

SEQ ID NO: 37 - protein, 19 aa, Homo sapiens
GIAPNLIRVTPACCITFVV

SEQ ID NO: 38 - protein, 27 aa, Homo sapiens
GFYKGIAPNLIRVTPACCITFVVYENV

SEQ ID NO: 39 - protein, 16 aa, Mus musculus
LVAGVSGGVLSNLALH

SEQ ID NO: 40 - protein, 26 aa, Mus musculus
YENLVAGVSGGVLSNLALHPLDLVKI

SEQ ID NO: 41 - protein, 24 aa, Mus musculus
LYQGVTPNVWGAGLSWGLYFFFYN

SEQ ID NO: 42 - protein, 27 aa, Mus musculus
GLYQGVTPNVWGAGLSWGLYFFFYNAI

SEQ ID NO: 43 - protein, 21 aa, Mus musculus
YLVSAAEAGAMTLCITNPLWV

SEQ ID NO: 44 - protein, 23 aa, Mus musculus
EYLVSAAEAGAMTLCITNPLWVT

SEQ ID NO: 45 - protein, 23 aa, Mus musculus
GLYKGFVPGLFGTSHGALQFMAY

SEQ ID NO: 46 - protein, 27 aa, Mus musculus
RGLYKGFVPGLFGTSHGALQFMAYELL

SEQ ID NO: 47 - protein, 22 aa, Mus musculus
AEYISVAALSKIFAVAATYPYQ

SEQ ID NO: 48 - protein, 29 aa, Mus musculus
QLSTAEYISVAALSKIFAVAATYPYQVVR

SEQ ID NO: 49 - protein, 19 aa, Mus musculus
GIAPNLIRVTPACCITFVV

SEQ ID NO: 50 - protein, 27 aa, Mus musculus
GFYKGIAPNLIRVTPACCITFVVYENV

SEQ ID NO: 51 - DNA, 975 bp, Danio rerio
  CDS (1)..(972)
atgaccgctaccatttccaggcagaggcacgccgcagtcgctgcagactcttctggctcttctttttccattacagcaaacctcctgcaactttcaaaacacatcaaatatgagaatcttgcagccggactcgctggtggtgttatttccacaatggtgctacatccattggatttgatcaaaatcaggtttgcagtaagtgatggtctgaaaatgaggccccaatacgatggcatgttagactgcatgaagaccatctggaagctggaaggcattagaggtctctatcagggagtgacgcccaacatctggggggccggatcatcatggggcctctacttcctcttttataatgctattaaagcatacacacaggagggacggcaaacagagctgagtgcatgtgaacacctggtgtccgcagcggaggcaggcattctgacgctttgcctcaccaatccagtctgggtgacaaagacccggctggtgctgcagtacaatgcagacccttcacggaagcagtacaagggaatgatggacgccctcgtgaaaatataccgtcacgagggtatcccaggactatacaggggttttgtgcctgggctggtcgggacttcccatgctgcactgcagttcatgacctatgaagggctaaaaagagagcagaacaaatgcaagaagatgccctctgaatccctgctgtccccattggaatacatcgccatagcagccatatccaaaatattcgctgtagcagtaacatacccctatcaggtggtccgcgctcgcctgcaggaccagcacaacaactacagtggaatagtggatgtcatgagaaggacctggagcaacgaaggggtggagggcttttacaaagggatggtgccaaacctggtccgagtcattcctgcgtgctgcatcaccttcctggtgttcgaaaatgtgtcacgcttgcttttgggcgagtatcactaa

SEQ ID NO: 52 - protein, 324 aa, Danio rerio
MTATISRQRHAAVAADSSGSSFSITANLLQLSKHIKYENLAAGLAGGVISTMVLHPLDLIKIRFAVSDGLKMRPQYDGMLDCMKTIWKLEGIRGLYQGVTPNIWGAGSSWGLYFLFYNAIKAYTQEGRQTELSACEHLVSAAEAGILTLCLTNPVWVTKTRLVLQYNADPSRKQYKGMMDALVKIYRHEGIPGLYRGFVPGLVGTSHAALQFMTYEGLKREQNKCKKMPSESLLSPLEYIAIAAISKIFAVAVTYPYQVVRARLQDQHNNYSGIVDVMRRTWSNEGVEGFYKGMVPNLVRVIPACCITFLVFENVSRLLLGEYH

SEQ ID NO: 53 - DNA, 969 bp, Drosophila melanogaster
  CDS (1)..(969)
atgaatccgatcaaggcacagtcaacgggcagtcccaagaaattcaacgtattcgcacacgtcaagtacgagcatttggttgccggagtatccggcggagtggtgtccacactcattctacatcccctggatttgatcaagattcgattcgcagttaacgatggccggacagctacggtgccgcaataccggggactgagcagcgccttcaccacgattttccggcaagagggcttccgcggactctacaaaggcgtcacccccaatgtctggggatcgggctcctcttggggcctgtacttcatgttctacaacaccattaagacatttatccaaggaggaaacacgaccatgccattgggccccacaatgaacatgcttgcagctgctgagtcgggaattctcaccctgctgctgaccaaccccatctgggtggtgaagacgcgtctctgcctgcagtgcgatgccgcgagtagtgccgagtacaggggcatgatccacgccttgggccagatatacaaggaggagggaatccgtggcctgtaccgcggctttgttcccggcatgttgggcgtctcccacggagccatccagttcatgacctacgaggagctgaagaacgcctacaacgaatatcgcaaactgcccatcgacacgaagctggccaccaccgagtacttggccttcgcggctgtctccaagctgatcgcagcggcggccacctacccgtaccaggtggtccgggcacggctgcaggaccaccatcaccgatacaacggcacctgggactgcatcaaacagacttggaggtttgagggctacagaggcttctacaaggggctgaaggcgagtttaacccgagtagtgcctgcctgcatggtcacctttctggtgtacgagaacgtctcgcatttcctgctcgccaggcggaagcgaattgagactaaagaggatgcgtcggacgtgtga

SEQ ID NO: 54 - protein, 322 aa, Drosophila melanogaster
MNPIKAQSTGSPKKFNVFAHVKYEHLVAGVSGGVVSTLILHPLDLIKIRFAVNDGRTATVPQYRGLSSAFTTIFRQEGFRGLYKGVTPNVWGSGSSWGLYFMFYNTIKTFIQGGNTTMPLGPTMNMLAAAESGILTLLLTNPIWVVKTRLCLQCDAASSAEYRGMIHALGQIYKEEGIRGLYRGFVPGMLGVSHGAIQFMTYEELKNAYNEYRKLPIDTKLATTEYLAFAAVSKLIAAAATYPYQVVRARLQDHHHRYNGTWDCIKQTWRFEGYRGFYKGLKASLTRVVPACMVTFLVYENVSHFLLARRKRIETKEDASDV

SEQ ID NO: 55 - DNA, 23 bp, Artificial Sequence
  Description of Artificial Sequence Synthetic primer
atggactacggggactttatcaa

SEQ ID NO: 56 - DNA, 21 bp, Artificial Sequence
  Description of Artificial Sequence Synthetic primer
accgacgccttctttccaact

SEQ ID NO: 57 - DNA, 18 bp, Artificial Sequence
  Description of Artificial Sequence Synthetic primer
ggcggccactacatcacg

SEQ ID NO: 58 - DNA, 20 bp, Artificial Sequence
  Description of Artificial Sequence Synthetic primer
tgcctcaagaacatagactg

SEQ ID NO: 59 - DNA, 30 bp, Artificial Sequence
  Description of Artificial Sequence Synthetic primer
atgaccgctaccatttccaggcagaggcac

SEQ ID NO: 60 - DNA, 30 bp, Artificial Sequence
  Description of Artificial Sequence Synthetic primer
ttagtgatactcgcccaaaagcaagcgtga

SEQ ID NO: 61 - protein, 22 aa, Drosophila melanogaster
FYKGLKASLTRVVPACMVTFLV

SEQ ID NO: 62 - protein, 24 aa, Drosophila melanogaster
GFYKGLKASLTRVVPACMVTFLVY

SEQ ID NO: 63 - DNA, 18 bp, Artificial Sequence
  Description of Artificial Sequence Synthetic mSOUP gene specific primer
ggcggccactacatcacg

SEQ ID NO: 64 - DNA, 45 bp, Artificial Sequence
  Description of Artificial Sequence Synthetic mSOUP gene specific primer
ctacttgtcatcatcgtccttgtagtcgctcactttcttttctct

SEQ ID NO: 65 - DNA, 19 bp, Artificial Sequence
  Description of Artificial Sequence Synthetic mSOUP gene specific primer
ctctgtcgcagcgctatcc

SEQ ID NO: 66 - DNA, 19 bp, Artificial Sequence
  Description of Artificial Sequence Synthetic mSOUP gene specific primer
gaaggcgggctctcacaac

SEQ ID NO: 67 - DNA, 29 bp, Artificial Sequence
  Description of Artificial Sequence Synthetic mSOUP gene specific primer
aatatttgccgtagcagcaacatacccgt

SEQ ID NO: 68 - protein, 18 aa, Artificial Sequence
  Description of Artificial Sequence Synthetic peptide
CYENVSHFLYDLREKKVS

SEQ ID NO: 69 - protein, 23 aa, Drosophila melanogaster
FYKGLVPYLVHVTPNICMPASFH

SEQ ID NO: 70 - protein, 25 aa, Drosophila melanogaster
GFYKGLVPYLVHVTPNICMPASFHL

SEQ ID NO: 71 - protein, 23 aa, Drosophila melanogaster
LTRVVPACMVTFLVYENVSHFLL

SEQ ID NO: 72 - protein, 26 aa, Drosophila melanogaster
ASLTRVVPACMVTFLVYENVSHFLLA

(19) United States
(12) Patent Application Publication
(10) Pub. No.: US 2008/0226638 A1
(43) Pub. Date: Sep. 18, 2008

(54) Modifier of Organelle Metabolism

(75) Inventors: Arnd Steuernagel, Gottingen (DE); Gunter Bronner, Randersacker (DE); Cord Dohrmann, Gottingen (DE); Thomas Ciossek, Ravensburg (DE); Roland Wehr, Gottingen (DE); Bettina Rudolph, Basel (CH); Dorothea Rudolph, Vienna (AT)

Correspondence Address: SUTHERLAND ASBILL & BRENNAN LLP, 999 PEACHTREE STREET, N.E., ATLANTA, GA, 30309, US

(73) Assignee: DEVELOGEN AG FUR ENTWICKLUNGSBIOLOGISCHE FORSCHUNG, Gottingen (DE)

(21) Appl. No.: 11/620,372

(22) Filed: Jan. 5, 2007

Related U.S. Application Data
(63) Continuation-in-part of application No. 10/432,737, filed on Oct. 14, 2003, now abandoned.

(30) Foreign Application Priority Data
Nov. 23, 2000 (EP) 00 125 693.2

(51) Int. Cl. A61K 39/395; A61K 31/70; A61P 1/00

(52) U.S. Cl. 424/139.1; 514/44

(57) ABSTRACT

Provided herein are methods for the treatment of metabolism disorders by delivering a therapeutically effective amount of a modulator of a SOUP1 polypeptide. The invention also relates to a nucleic acid molecule encoding a polypeptide contributing to membrane stability and/or function of organelles.

### CROSS REFERENCES TO RELATED APPLICATIONS

This application is a continuation-in-part of U.S. application Ser. No. 10/432,737 filed on May 23, 2003, pending, which claims priority to European Application No. 00 125 693.2 filed on Nov. 23, 2000 and PCT Patent Application No. PCT/EP01/13663 filed on Nov. 23, 2001.

### FIELD OF THE INVENTION

The present invention relates to a nucleic acid molecule encoding a polypeptide contributing to membrane stability and/or function of organelles, wherein said nucleic acid molecule (a) hybridizes under herein defined stringent conditions to the complementary strand of a nucleic acid molecule encoding the amino acid sequence of disclosed herein; (b) hybridizes under herein defined conditions to the complementary strand of a nucleic acid molecule as disclosed herein; (c) it is degenerate with respect to the nucleic acid molecule of (a); (d) encodes a polypeptide which comprises at least one, preferably at least two, more preferably at least three, more preferably at least four, more preferably at least five and most preferably six amino acid sequences described herein as putative transmembrane regions; (e) encodes a polypeptide which is at least 85%, preferably at least 90%, more preferably at least 95%, more preferably at least 98% and up to 99.6% identical to a herein disclosed amino acid sequence representing a polynucleotide contributing to membrane stability and/or function of organelles; (f) encodes a polypeptide which is at least 90%, preferably at least 95%, more preferably at least 98% and up to 99.6% identical to a herein disclosed amino acid sequence representing a polynucleotide contributing to membrane stability and/or function of organelles; (g) encodes a polypeptide which is at least 35%, preferably at least 50%, more preferably at least 60%, more preferably at least 70%, more preferably at least 80%, more preferably at least 90%, most preferably at least 95% and most preferably at least 99% identical to the amino acid sequence disclosed herein; (h) differs from the nucleic acid molecule of (a) to (g) by mutation and wherein said mutation causes an alteration, deletion, duplication or premature stop in the encoded polypeptide or (i) has a sequence as depicted herein. Furthermore, the invention provides for vectors comprising said nucleic acid molecule as well as to hosts transformed with said vector. The invention also relates to polypeptides encoded by said nucleic acid molecules and to antibodies, fragments or derivatives thereof or an aptamer or another receptor specifically recognizing the nucleic acid molecule or the polypeptide of the invention. The invention also describes compositions comprising nucleic acid molecules, vectors, hosts, polypeptides, fusion proteins, antibodies, fragment or derivative thereof or aptamers or other receptors or anti-sense oligonucleotides of the invention. Preferably these compositions are diagnostic compositions or pharmaceutical compositions. Furthermore, the invention provides for methods of identifying a polypeptide or (a) substance(s) involved in cellular metabolism in an animal or an plant or capable of modifying homeostasis and for identifying a polypeptide involved in the regulation of body weight in a mammal. The invention also relates to methods of identifying a compound influencing the expression of the nucleic acid molecule or the polypeptide of the invention. In addition, methods are disclosed for assessing the impact of the expression of one or more compounds of the invention. Finally, the invention provides for compositions comprising inhibitors and/or stimulators of the (poly)peptide of the invention and it provides for kits comprising the compounds of the invention.

### BACKGROUND OF THE INVENTION

Mitochondria are the energy suppliers of animal cells. Most of the energy available from metabolising foodstuffs like carbohydrates, fats etc. is used to create a proton gradient across the inner mitochondrial membrane. This proton gradient drives the enzyme ATP synthetase that produces ATP, the cells major fuel substance (Mitchell P, Science 206, 1979, 1148-1159). In the mitochondria of brown adipose tissue exists a protein (Uncoupling Protein 1) that tunnels protons through the inner mitochondrial membrane (review in Klingenberg M, Huang S G, Biochim Biophys Acta 1999, 1415(2):271-96). The energy stored in the proton gradient is thereby released as heat and not used for ATP synthesis.

When the energy intake of an animal exceeds expenditure surplus energy can be stored as fat in adipose tissue. The generation of a proton leak across the inner mitochondrial membrane by the activation of uncoupling proteins would reduce caloric efficiency and thus avoid the accumulation of excess body fat (obesity) that is detrimental to the animals health. In human, however, brown adipose tissue is almost absent in adults. Therefore UCP1 was not considered to be a major factor in the formation or prevention of human obesity. Recently the discovery of further proteins of similar sequence (UCP2-UCP5) that are widely expressed in human tissues (e.g. white adipose tissue, muscle) made this members of the UCP family to important targets for pharmaceutical research (reviewed in Adams S H, Nutr 2000, 130(4):711-4). Interestingly, and as reviewed in Ricquier, Biochem J. 345 (2000), 161-179, further homologues have been identified, like, inter alia, the plant UCPs StUCP (from Solanum tuberculosum) and AtUCP (Arabidopsis thaliana). Although the in vivo function of these proteins is still unknown, the possibility to influence UCP activity would be a conceivable therapy for the treatment or prevention of obesity and related diseases.

Mitochondria have a very specialized function in energy conversion and said function is reflected in their morphological structure, namely the distinct internal membrane. This internal membrane does not only provide the framework for electron-transport processes but also creates a large internal compartment in each organelle in which highly specialized enzymes are confined. Therefore, there is a strong relationship between mitochondrial energy metabolism and the biochemical/biophysical properties of these organelles.

The technical problem underlying the invention was to provide for means and methods for modulating the biological/biochemical activities of mitochondria and, thereby, modulating metabolic conditions in eukaryotic cells which influence energy expenditure, body temperature, thermogenesis, cellular metabolism to an excessive or deficient supply of substrate(s) in order to regulate the ATP level, the NAD⁺/NADH ratio, and/or superoxide production.

### SUMMARY OF THE INVENTION

The solution to this technical problem is achieved by providing the embodiments characterized in the claims.

Accordingly, the present invention relates to a nucleic acid molecule encoding a polypeptide contributing to membrane stability and/or function of organelles, wherein said nucleic acid molecule

(a) hybridizes at 65° C. in a solution containing 0.2×SSC and 0.1% SDS to a nucleic acid molecule encoding the amino acid sequence of SEQ ID NO: 8, of SEQ ID NO: 54, of SEQ ID NO. 10, SEQ ID NO: 12, SEQ ID NO: 14 and/or SEQ ID NO: 52 and/or the complementary strand thereof;

(b) hybridizes at 65° C. in a solution containing 0.2×SSC and 0.1% SDS to a nucleic acid molecule as depicted in SEQ ID NO: 6, 7 or 53, SEQ ID NO: 9, SEQ ID NO: 11, SEQ ID NO: 13 and/or SEQ ID NO: 51 and/or the complementary strand thereof.

(c) it is degenerate with respect to the nucleic acid molecule of (a) or (b);

(d) encodes a polypeptide which comprises at least one, preferably at least two, more preferably at least three, more preferably at least four, more preferably at least five and most preferably six amino acid sequences as depicted in any one of SEQ ID NOs: 15 to 50, 61 and 62;

(e) encodes a polypeptide which is at least 85%, preferably at least 90%, more preferably at least 95%, more preferably at least 98%, and up to 99.6% identical to SEQ ID NO: 8;

(f) encodes a polypeptide which is at least 90%, preferably at least 95%, more preferably at least 98% and up to 99.6% identical to SEQ ID NO: 54;

(g) encodes a polypeptide which is at least 35%, preferably at least 50%, more preferably at least 60%, more preferably at least 70%, more preferably at least 80%, more preferably at least 90%, most preferably at least 95% and most preferably at least 99% identical to the amino acid sequence as depicted in any one of SEQ ID NOs: 10, 12, 14 or 52;

(h) differs from the nucleic acid molecule of (a) to (g) by mutation and wherein said mutation causes an alteration, deletion, duplication or premature stop in the encoded polypeptide; or

(i) has the sequence as depicted in SEQ ID NOs: 9, 11, 13 or 51.

Preferably, the nucleic acid molecule is a mammalian SOUP nucleic acid molecule, more particularly a human SOUP molecule. Specific examples are nucleic acid molecules in SEQ ID NO: 9 (human SOUP 1), SEQ ID NOs: 11 and 13 (mouse SOUP1) and SEQ ID NO: 51 (Danio rero SOUP1). The nucleic acid molecule preferably encodes mammalian SOUP1 protein, particulary a human SOUP protein. Specific examples of preferred polypeptides have the amino acid sequence of SEQ ID NO: 10 (human SOUP 1), SEQ ID NOs: 12 or 14 (mouse SOUP1) and SEQ ID NO: 52 (Danio rero SOUP1).

The present invention particularly refers to the modulation of SOUP nucleic acids and/or proteins, particularly in mammalian cells and/or organisms. More preferably, the invention refers to the modulation of human SOUP.

### BRIEF DESCRIPTION OF THE DRAWINGS

The figures show:

FIG. 1 shows the nucleotide and amino acid sequence of Drosophila UCPy. Shown are the full length cDNA (SEQ ID NO:3), the open reading frame (SEQ ID NO:4), and the deduced amino acid sequence (SEQ ID NO:5).

FIG. 2 shows the overexpression of Drosophila UCPy in the eye. In the fly shown on the left part of the picture, the dUCPy expression is normal, and the eye is normally developed. In the fly shown on the right part of the picture, dUCPy was overexpressed, and the phenotype observed shows a reduction of the fly eye. If SOUP1 is co-expressed in this phenotype, the eye defect can be rescued. The eye of such rescue flies (not shown) is almost identical to the normal eye of the left fly.

FIG. 3a. shows the cDNA of Drosophila melanogaster for Accession Number GH22139. Shown are the full length cDNA nt 1-2953 (SEQ ID NO:6), the open reading frame (SEQ ID NO:7), and the deduced amino acid sequence (SEQ ID NO:8).

FIG. 3b. shows the cDNA of Drosophila melanogaster for the splice variant of Accession Number GH22139. Shown are the dSOUP-sp1 open reading frame (nt 688-1497 and 2482-2641 of SEQ ID NO:6), and the deduced amino acid sequence for the SOUP splice variant dSOUP-sp1.

FIG. 3c. shows the gene prediction from the SOUP1 locus in public databases. Shown are the open reading frame of splice variant dSOUP-CG8026 (nt 688-1569, 2280-2320 and 2482-2641 of SEQ ID NO:6), and the deduced amino acid sequence for splice variant dSOUP-CG8026, as predicted from public databases.

FIG. 4 shows the transmembrane domain plot of SOUP1 proteins.

FIG. 4a shows the transmembrane domain plot for Drosophila SOUP1,

FIG. 4b shows the transmembrane domain plot for human SOUP1-CG80267,

FIG. 4c shows the transmembrane domain plot for human SOUP1,

FIG. 4d shows the transmembrane domain plot for mouse SOUP1,

FIG. 4e shows the transmembrane domain plot for zebrafish SOUP1.

FIG. 5 shows the transmembrane domains of SOUP1 variants.

FIG. 5a shows the comparison of different Drosophila SOUP1 variants. The nucleotide numbering relates to the sequence in SEQ ID NO: 6. The transmembrane domains of splice varians dSOUP1 and dSOUP1-sp1 are different. Splice variant dSOUP1-CG8026 would generate a protein with seven transmembrane domains.

The transmembrane domains in dSOUP1, variant dSOUP1-sp1, variant dSOUP1-CG8026, human SOUP1 (hSOUP1), and mouse (mSOUP1) are shown in FIGS. 5b, 5c, 5d, 5e and 5f, respectively.

FIG. 6. shows human SOUP1. Shown are the open reading frame (SEQ ID NO:9), and the deduced amino acid sequence for human SOUP (SEQ ID NO: 10).

FIG. 7. shows mouse SOUP1. Shown are the open reading frame (SEQ ID NO: 11), and the deduced amino acid sequence for mouse SOUP (SEQ ID NO: 12).

FIG. 8. shows the alternative mouse gene 61. Shown are the open reading frame (SEQ ID NO: 13), and the deduced amino acid sequence for mouse 61 (SEQ ID NO: 14).

FIG. 9. shows Danio rero SOUP1. Shown are the open reading frame (SEQ ID NO:51), and the deduced amino acid sequence for mouse SOUP (SEQ ID NO:52).

FIG. 10 shows the expression of UCP2 in a beta-actin-mSOUP-flg transgenic mouse model in vivo.

FIG. 10 a shows the northern analysis,

FIG. 10 b shows the quantification of the data shown in FIG. 10a.

FIG. 11 shows the localization of SOUP1 in mitochondria of NIH 3T3 cells. NIH 3T3 cells were transiently transfected with an expression vector for mouse Soup, fixed und immunostained with an antisera against mouse Soup (see Example 13).

FIG. 12 shows the effect of SOUP1 knock down by RNA interference in 3T3-L1 adipocyte cells. The triglyceride contents (μmol triglyceride/mg protein) in cells treated with the RNAi construct (DG175 RNAi) and with a control (pLPCX) are shown.

### DETAILED DESCRIPTION OF THE INVENTION

As documented in the appended examples, the present invention provides for genes and gene products which are either directly or indirectly involved in membrane stability and/or function of organelles, in particular of mitochondria.

Further, the examples demonstrate that a knock down of the SOUP1 transcript by RNA interference leads to a decrease of the triglyceride content in adipocytes. In contrast thereto, overexpression of SOUP1 leads to an increased triglyceride accumulation.

The term “membrane stability” as used herein comprises not only the overall stability but also comprises local stabilities on membranes of organelles, for example of the inner and outer membrane, but in particular of the inner membrane. The term “membrane stability” relates, therefore, to structural features of the membranes, provided by protein-protein interactions as well as by protein-lipid interactions leading to a defined membrane composition.

The term “contributing to membrane function of organelles” as employed herein above relates to functions of the above defined polypeptide comprising, inter alia, transport functions (like active and passive transport of ions, metabolites, vitamines, etc.), regulator functions of other membrane proteins (like transporters, carriers) or modifier functions of other (membrane) proteins (like enhancement/suppressor functions) and/or other functions as defined herein below.

The term “organelles” as employed herein not only relates to mitochondria but also to further organelles, like peroxisomes or plant cell organelles, e.g. chloroplasts.

The terms “hybridizes” and “hybridizing” as employed in context of the present invention preferably relate to stringent conditions as, inter alia, defined herein above, e.g. 0.2×SSC, 0.1% SDS at 65° C. Said conditions comprise hybridization conditions and particularly washing conditions. It is preferred that washing conditions are more stringent than hybridization conditions. By setting the conditions for hybridization, the person skilled in the art can determine if strictly complementary sequences or sequences with a higher or lower degree of homology are to be detected. The setting of conditions is well within the skill of the artisan and to be determined according to protocols described, for example, in Sambrook, Molecular Cloning, A Laboratory Manual, 2^{nd} edition (1989), Cold Spring Harbor Laboratory Press, Cold Spring Harbor, N.Y. or Hames and Higgins, “Nucleic acid hybridization, a practical approach”, IRL Press, Oxford (1985). Non-stringent hybridization conditions for the detection of homologous and not exactly complementary sequences may be set at 6×SSC, 1% SDS at 65° C.

The molecules hybridizing to the nucleic acid molecules of the invention also comprise fragments, derivatives and allelic variants of the above-described nucleic acid molecules which encode (poly)peptides regulating, causing or contributing to obesity described in the present invention. In this regard, fragments are defined as parts of the nucleic acid molecules, which are long enough in order to encode said (poly)peptides. The term derivatives means that the sequences of these hybridizing molecules differ from the sequences of the above-mentioned nucleic acid molecules at one or more positions and that they exhibit a high degree of homology to these sequences. For example, homology means a sequence identity to sequences as identified in SEQ ID NOs: 10, 12, 14 or 52 of at least 35%, in particular an identity of at least 45%, preferably of more than 50%, more preferably more than 60%, more preferably more than 70%, more preferably more than 80% and still more preferably a sequence identity of more than 90%. Considering a sequence identified as SEQ ID NO: 8 said homology means a sequence identity of at least 85%. For the sequence depicted in SEQ ID NO: 54, a sequence identity of at least 90% is considered “homologous”. The person skilled in the art may employ computer programs and packages in order to determine homology values. Generally, nucleotide or amino acid sequence identities/homologies can be determined conventionally by using known computer programs such as BLASTIN, BLASTP, NALIGN, PALIGN or b12seq using particular algorithms to find the best segment of homology between two segments.

As shown in the appended examples, in the context of the present invention the comparative analysis of the percentage of identities at the amino acid level are preferably obtained using the “b12seq” program from NCBI using the following parameters: Open Gap Cost. 11 and Gap Extension Cost: 1. However, the program allows any positive integer for said value(s).

Furthermore, in the context of the present invention comparative analysis of the percentage of identities at the nucleotide level of the different nucleic acid sequences, on the other hand, are preferably obtained using the “matcher” program of the EMBOSS package using the following parameters: Gap penalty value: 16 and Gap length value: 4 alternatively, the program accepts any positive integer. It was found that these parameters are the best suited to calculate the percentage of identity over the reference nucleotide or amino acid sequences, especially considering the different levels of homology among the different sequences analyzed. Any deviations occurring when comparing with the above-described nucleic acid molecules may be caused by deletion, substitution, insertion or recombination.

Moreover, homology means that functional and/or structural equivalence exists between the respective nucleic acid molecules or the proteins they encode. The nucleic acid molecules, which are homologous to the above-described molecules and represent derivatives of these molecules, are generally variations of these molecules that constitute modifications which exert the same biological function. These variations may be naturally occurring variations, for example sequences derived from other organisms, or mutations, whereby these mutations may have occurred naturally or they may have been introduced by means of a specific mutagenesis. Moreover, the variations may be synthetically produced sequences. The allelic variants may be naturally occurring as well as synthetically produced variants or variants produced by recombinant DNA techniques.

The proteins encoded by the various variants of the nucleic acid molecules according to the invention exhibit certain common characteristics. Biological activity, molecular weight, immunological reactivity, conformation etc. may belong to these characteristics as well as physical properties such as the mobility in gel electrophoresis, chromatographic characteristics, sedimentation coefficients, solubility, spectroscopic properties, stability, pH-optimum, temperature-optimum etc.

Preferably, the above described nucleic acid molecules encode a polypeptide comprising at least one, most preferably at least six amino acid sequences as depicted in any one of SEQ ID NOs: 15 to 50, 61 and 62. Said SEQ ID NOs relate to predicted transmembrane regions/domains and are also described in the appended examples and figures. Sequences as shown in SEQ ID NOs: 15 to 26, 61 and 62 relate to transmembrane regions as deduced in a nucleic acid molecule of the invention (and its corresponding amino acid sequence) which is obtainable from Drosophila melanogaster, SEQ ID NOs: 27 to 38 relate to transmembrane domains as deduced in a nucleic acid molecule obtainable from Homo sapiens and SEQ ID NOs: 39 to 50 relate to transmembrane domains of the protein as defined herein and deducible from Mus musculus. Within the scope of the present invention are therefore also polypeptides encoded by the nucleic acid molecule of the invention which comprise at least one transmembrane domain as shown in any one of SEQ ID NOs: 15 to 50, 61 and 62. The present invention, however, also comprises constructs wherein transmembrane regions/domains from different species are artificially linked. It is most preferred that the nucleic acid molecule as defined in alternative (d) herein above encodes for a polypeptide comprising at least one, more preferably at least six amino acid sequences as depicted in any one of SEQ ID NOs: 27 to 50, deduced from mouse and human. Yet, preferred combinations comprise at least one and preferably all transmembrane regions from one species.

It is preferred that the nucleic acid molecule of the invention encodes a polypeptide contributing to membrane stability and/or function of organelles which is at least 85% and up to 99.6% identical to the amino acid sequence as depicted in SEQ ID NO: 8 or which is at least 90% and up to 99.6% identical to the amino acid sequence as depicted in SEQ ID NO: 54. SEQ ID NO: 8 depicts a polypeptide encoded, inter alia, by SEQ ID NO: 7 and represents a protein of Drosophila which has surprisingly been found to be involved in membrane function and/or stability of organelles and has, in particular, be found to be able to modify UCPS; see also appended examples. The amino acid sequence as depicted in SEQ ID NO: 54 (encoded, inter alia, by SEQ ID NO: 53) comprises a splice variant of the above described protein. As demonstrated in the appended examples, the here described polypeptide (and encoding nucleic acid molecule) was able to modify, e.g. suppress a specific eye phenotype in Drosophila which was due to the overexpression of the Drosophila melanogaster gene dUCPy. The overexpression of dUCP (with homology to human UCPs) in the compound eye of Drosophila led to a clearly visible eye defect (see appended examples and figures) which can be used as a “read-out” for a genetical “modifier screen”.

In said “modifier screen” thousands of different genes are mutagenized to activate their expression in the eye. Should one of the mutagenized genes interact with dUCPy and modify its activity an enhancement or suppression of the eye defect will occur. Since such flies are easily to discern they can be selected to isolate the interacting gene.

As shown in the appended examples, a gene was deduced that can suppress the eye defect induced by the activity of dUCPy. This gene is called Suppressor Of Uncoupling Protein 1 (SOUP1).

The present invention also relates to SOUP1 proteins encoded by the nucleic acid molecule of the invention and depicted in SEQ ID NOs: 10, 12, 14 and 52. It is preferred that the encoded polypeptide is at least 35% and most preferably at least 99% identical to the amino acid sequence as depicted in any one of SEQ ID NOs: 10, 12, 14 or 52. SEQ ID NO: 10 depicts the human homologue of SOUP1, SEQ ID NO: 12 and 14 depict two variants of the mouse SOUP1, SEQ ID NO: 52 depicts SOUP1 of the zebrafish (Danio rerio).

It is envisaged that mutations in the herein described SOUP1-polypeptides (and genes) lead to phenotypic and/or physiological changes which may comprise a modified and altered mitochondrial activity. This, in turn, may lead to, inter alia, an altered energy metabolism, altered thermogenesis and/or altered energy homeostasis.

In a preferred embodiment the above described nucleic acid molecule of the invention is DNA. In this context it is understood that the term “nucleic acid molecule” comprises coding and, wherever applicable, non-coding sequences, like, inter alia, 5′ and 3′ non-coding sequences. Said 5′ and/or 3′ non-coding regions may comprise (specific) regulatory sequences ensuring initiation of transcription and optionally poly-A signals ensuring termination of transcription and/or stabilization of the transcript. Additional 5′ and 3′ non-coding regions may comprise promoters and/or transcriptional as well as translational enhancers. Furthermore, the term “nucleic acid molecule” may comprise intron(s) and splice variants, where applicable.

The term DNA as used herein comprises, inter alia, single-stranded or double-stranded DNA, e.g., synthetic DNA, cDNA and genomic DNA. Furthermore, the nucleic acid molecule of the invention may also be a RNA molecule such as mRNA. In accordance with the present invention, the term “nucleic acid molecule” comprises also any feasible derivative of a nucleic acid to which a nucleic acid probe may hybridize. Said nucleic acid probe itself may be a derivative of a nucleic acid molecule capable of hybridizing to said nucleic acid molecule or said derivative thereof. The term “nucleic acid molecule” further comprises peptide nucleic acids (PNAs) containing DNA analogs with amide backbone linkages (Nielsen, Science 254 (1991), 1497-1500).

In this context it has to be stressed that nucleic acid molecules of the invention may also be chemically synthesized, using, inter alia, synthesizers which are known in the art and commercially available, like, e.g. the ABI 394 DNA-RAN-synthesizers.

It is preferred that the nucleic acid molecule of the invention encodes a polypeptide contributing to membrane stability and/or function of organelles, wherein said polypeptide contributing to membrane stability and/or function in organelles is expressed in mitochondria and/or peroxisomes. It is particularly preferred that said polypeptide participates in the maintenance of said membrane.

Furthermore, it is envisaged that the nucleic acid molecule of the invention encodes a polypeptide, wherein said polypeptide contributing to membrane stability and/or function in organelles is a transporter molecule and/or a regulator of a transporter molecule. It is, e.g., envisaged that the polypeptide encoded by the nucleic acid molecule of the invention regulates, directly or indirectly, carrier and/or transport molecules capable of transporting molecules like ions, metabolites or vitamins across membranes and/or that said polypeptide is such a transporter/carrier molecule.

It is particularly preferred that the nucleic acid molecule the invention encodes a polypeptide as defined herein above, wherein said polypeptide is a modifying polypeptide. Particularly preferred modifying polypeptides comprise modifiers of mitochondrial proteins, for example the modification of a member of the UCP family.

Said member(s) of the UCP (uncoupling protein) family are known in the art and comprise, UCP1, UCP2, UCP3, UCP4, UCP5, StUCP or AtUCP, see, inter alia, Ricquier (2000), loc. cit. The above mentioned modification of mitochondrial proteins, and in particular of UCPs, may occur by direct interaction with said protein or, also, by supplying/importing/exporting ions, metabolites or vitamins and the like (or by blocking these processes) which are necessary for the function or activity of said mitochondrial protein or which are generated by the activity of said mitochondrial protein. Therefore, said “modification” also relates to transport- and supply-phenomena. Furthermore, said “modification” comprises the control of the function of one or more proteins/polypeptides, preferably of members of the UCP family. Most preferred are “modifications” comprising events which influence the metabolism of the cell, in particular the energy metabolism.

The present invention relates also, as pointed out herein above, to “variants” of the nucleic acid molecules described herein.

The term “variant” means in this context that the nucleotide and their encoded amino acid sequence, respectively, of these polynucleotides differs from the sequences of the above-described nucleic acid molecules and (poly)peptides contributing to membrane stability and/or function of organelles in one or more nucleotide positions and are highly homologous to said nucleic acid molecules. Homology is understood as defined herein above. The deviations from the sequences of the nucleic acid molecules described above can, for example, be the result of nucleotide substitution(s), deletion(s), addition(s), insertion(s) and/or recombination(s). Homology can further imply that the respective nucleic acid molecules or encoded proteins are functionally and/or structurally equivalent. The nucleic acid molecules that are homologous to the nucleic acid molecules described above and that are derivatives of said nucleic acid molecules are, for example, variations of said nucleic acid molecules which represent modifications having the same biological function, in particular encoding proteins with the same or substantially the same biological function. They may be naturally occurring variations, such as sequences from other mammals or mutations. The term “variants” in this context furthermore comprises, inter alia, allelic variations or splice variants as described herein above. Naturally occurring SOUP1 protein or soup1 gene variants are called “allelic variants”, and refer to one of several alternate forms of a gene occupying a given locus on a chromosome of an organism. (Genes II, Lewin, B., ed., John Wiley & Sons, New York (1985) and updated versions). These allelic variants can vary at either the polynucleotide and/or (poly)peptide level. Alternatively, non-naturally occurring variants may be produced by mutagenesis techniques or by direct synthesis. Using known methods of protein engineering and recombinant DNA technology, variants may be generated to improve or alter the characteristics of the herein described SOUP1 proteins/soup1 genes. Therefore, the term “allelic variant” also comprises synthetically produced or genetically engineered variants.

The nucleic acid molecule of the invention may be of natural origin, synthetic or semisynthetic or it may be a derivative.

The nucleic acid molecules of the invention encoding the above described (poly)peptides, e.g. wildtype and mutated forms of the SOUP1 and/or fragments thereof find a wide variety of applications including use as translatable transcripts, hybridization probes, PCR primers or the use in expression profiling of nucleic acids, for example on appropriately coated chips or in diagnostic and/or pharmaceutical settings. Useful PCR primers can be deduced by the person skilled in the art from the nucleic acid molecules of the invention. Particularly useful primers are, inter alia, the employed in the appended examples.

In particular they may be used in detecting the presence of soup1 genes and gene transcripts and in detecting and/or amplifying nucleic acids encoding further soup1 homologues or structural analogues. Given the probes, materials and methods disclosed herein, inter alia, for probing cDNA and genomic libraries, the person skilled in the art is in a position to recover corresponding homologues. As described herein below, the nucleic acid molecules of the invention may be part of specific expression vectors and may be incorporated into recombinant cells for expression and screening and in transgenic animals for functional studies (e.g. the efficacy of candidate drugs for disease associated with expression of SOUP1) as described herein below.

Furthermore, in diagnosis, specific hybridization probes related to the soup1 gene(s) as described herein and single nucleotide polymorphisms present in soup1 alleles find use in identifying wild-type and mutant soup1 alleles in clinical and laboratory samples. Mutant alleles are, inter alia, used to generate allele-specific oligonucleotide (ASO) probes for, e.g., high-throughput clinical diagnosis. For therapeutic approaches nucleic acid molecules of the invention as described herein above and herein below may be employed to modulate cellular expression or intracellular concentration or availability of active (poly)peptides of the invention. These nucleic acid molecules may comprise antisense molecules, i.e. single-stranded sequences comprising the complements of the disclosed nucleic acids of the invention.

The nucleic acid molecule(s) of the invention may be a recombinantly produced chimeric nucleic acid molecule comprising any of the aforementioned nucleic acid molecules either alone or in combination. Preferably, said nucleic acid molecule is part of a vector.

The present invention therefore also relates to a vector comprising the nucleic acid molecule of the present invention. The vector of the present invention may be, e.g., a plasmid, cosmid, virus, bacteriophage or another vector used e.g. conventionally in genetic engineering, and may comprise further genes such as marker genes which allow for the selection of said vector in a suitable host cell and under suitable conditions. Furthermore, the vector of the present invention may, in addition to the nucleic acid sequences of the invention, comprise expression control elements, allowing proper expression of the coding regions in suitable hosts. Such control elements are known to the artisan and may include a promoter, a splice cassette, translation initiation codon, translation and insertion site for introducing an insert into the vector. Preferably, the nucleic acid molecule of the invention is operatively linked to said expression control sequences allowing expression in eukaryotic or prokaryotic cells.

Control elements ensuring expression in eukaryotic and prokaryotic cells are well known to those skilled in the art. As mentioned herein above, they usually comprise regulatory sequences ensuring initiation of transcription and optionally poly-A signals ensuring termination of transcription and stabilization of the transcript. Additional regulatory elements may include transcriptional as well as translational enhancers, and/or naturally-associated or heterologous promoter regions. Possible regulatory elements permitting expression in for example mammalian host cells comprise the CMV-HSV thymikine kinase promoter, SV40, RSV-promoter (Rous sarcome virus), human elongation factor 1α-promoter, aPM-I promoter (Schaffer, Biochem. Biophys. Res. Commun. 260 (1999), 416-425), or inducible promoter(s), like, metallothionein or tetracyclin, or enhancers, like CMV enhancer or SV40-enhancer. For the expression in prokaryotic cells, a multitude of promoters including, for example, the tac-lac-promoter or the trp promoter, has been described. Besides elements which are responsible for the initiation of transcription such regulatory elements may also comprise transcription termination signals, such as SV40-poly-A site or the tk-poly-A site, downstream of the polynucleotide. In this context, suitable expression vectors are known in the art such as Okayama-Berg cDNA expression vector pcDV1 (Pharmacia), pRc/CMV, pcDNA1, pcDNA3 (In-vitrogene), pSPORT1 (GIBCO BRL), Casper, Casper-HS43, pUAST, or prokaryotic expression vectors, such as lambda gt11. Beside the nucleic acid molecules of the present invention, the vector may further comprise nucleic acid sequences encoding for secretion signals. Such sequences are well known to the person skilled in the art. Furthermore, depending on the expression system used leader sequences capable of directing the (poly)peptide to a cellular compartment may be added to the coding sequence of the nucleic acid molecules of the invention and are well known in the art. The leader sequencers) is (are) assembled in appropriate phase with translation, initiation and termination sequences, and preferably, a leader sequence capable of directing secretion of translated protein, or a protein thereof, into the periplasmic space or extracellular medium. Optionally, the heterologous sequence can encode a fusion protein including an C- or N-terminal identification peptide imparting desired characteristics, e.g., stabilization or simplified purification of expressed recombinant product. Once the vector has been incorporated into the appropriate host, the host is maintained under conditions suitable for high level expression of the nucleotide sequences, and, as desired, the collection and purification of the (poly)peptide(s) or fragments thereof of the invention may follow.

Furthermore, the vector of the present invention may also be a gene transfer or gene targeting vector. Gene therapy, which is based on introducing therapeutic genes into cells by ex-vivo or in-vivo techniques is one of the most important applications of gene transfer. Suitable vectors, methods or gene-delivering systems for in-vitro or in-vivo gene therapy are described in the literature and are known to the person skilled in the art; see, e.g., Giordano, Nature Medicine 2 (1996), 534-539; Schaper, Cire. Res. 79 (1996), 911-919; Anderson, Science 256 (1992), 808-813, Isner, Lancet 348 (1996), 370-374; Muhlhauser, Circ. Res. 77 (1995), 1077-1086; Onodua, Blood 91 (1998), 30-36; Verzeletti, Hum. Gene Ther. 9 (1998), 2243-2251; Verma, Nature 389 (1997), 239-242; Anderson, Nature 392 (Supp. 1998), 25-30; Wang, Gene Therapy 4 (1997), 393-400; Wang, Nature Medicine 2 (1996), 714-716; WO 94/29469; WO 97/00957; U.S. Pat. No. 5,580,859; U.S. Pat. No. 5,589,466; U.S. Pat. No. 4,394,448 or Schaper, Current Opinion in Biotechnology 7 (1996), 635-640, and references cited therein. In particular, said vectors and/or gene delivery systems are also described in gene therapy approaches in adipocyte (see, inter alia, U.S. Pat. No. 5,869,037 or Zhou, PNAS USA 96 (1999), 2391-2395) or in the hypothalamus (see, inter alia, Geddes, Front Neuroendocrinol. 20 (1999), 296-316 or Geddes, Nat. Med. 3 (1997), 1402-1404). The nucleic acid molecules and vectors of the invention may be designed for direct introduction or for introduction via liposomes, viral vectors (e.g. adenoviral, retroviral), electroporation, ballistic (e.g. gene gun) or other delivery systems into the cell. Additionally, a baculoviral system can be used as eukaryotic expression system for the nucleic acid molecules of the invention.

As will be discussed herein below, the nucleic acid molecule of the present invention and/or the above described vectors/hosts of the present invention may be particularly useful as pharmaceutical compositions. Said pharmaceutical compositions may be employed in diagnostic and/or therapeutic approaches, e.g. in gene therapy approaches. In this context, it is envisaged that the nucleic acid molecules and/or vectors of the present invention may be employed to modulate, alter and/or modify the cellular expression and/or intracellular concentration of the (poly)peptide(s) of the invention or of (a) fragment thereof. Said modulation, alteration and/or modification may lead to up- or downregulation of the SOUP1 (poly)peptide and/or the gene product of the herein described SOUP1 gene. Furthermore, said therapeutic approache(s) may lead to an alteration and/or modulation of the availability of active SOUP1 (poly)peptide/protein/gene product. In this context, the term “active” refers to the ability to perform its (normal) cellular function in an organism.

For gene therapy applications, nucleic acids encoding the (poly)peptide of the invention or fragments thereof may be cloned into a gene delivering system, such as a virus and the virus used for infection and conferring disease ameliorating or curing effects in the infected cells or organism.

As mentioned herein above, the nucleic acid molecule(s) and/or vector(s) may be employed in order to modulate/alter the gene expression or intracellular concentration of SOUP1 protein/(poly)peptide. Said modulation/alteration may also be achieved by antisense-approaches.

Antisense modulation of SOUP1 expression may employ antisense nucleic acids operably linked to gene regulatory sequences. For example, cells are transfected with a vector comprising an soup1 sequence with a promoter sequence oriented such that transcription of the gene yields an antisense transcript capable of binding to endogenous soup1 encoding mRNA. Transcription of the antisense nucleic acid may be constitutive or inducible and the vector may provide for stable extrachromosomal maintenance and integration. Alternatively, single-stranded antisense nucleic acids that bind to genomic DNA or mRNA encoding a (poly)peptide of the invention or a fragment thereof may be administered to the target cell, in or temporarily isolated from a host, at a concentration that results in a substantial reduction in expression of said (poly)peptide. Furthermore, it is envisaged that expression of the (poly)peptide of the invention may be influenced, suppressed by other means than antisense approaches. Therefore, reduced expression of the (poly)peptide of the invention may also be achieved by RNA-mediated gene interference, which applies double-stranded RNA instead of antisense nucleic acids (see, Sharp, Genes Dev. 13 (1999), 139-141). Gene suppression by double stranded RNA or RNAi-approach is also described in Hunter, Curr. Biol. 10 (2000), R137-R140.

The nucleic acid molecule of the invention may therefore be used for the construction of appropriate anti-sense oligonucleotides which are able to inhibit the function of the nucleic acid molecules which either encode wildtype or mutant versions of the SOUP1 (poly)peptide of this invention. Said anti-sense nucleotide comprises preferably at least 15 nucleotides, more preferably at least 20 nucleotides, even more preferably 30 nucleotides and most preferably at least 40 nucleotides.

In addition, ribozyme approaches are also envisaged in this invention. Ribozymes may specifically cleave the nucleic acid molecule of the invention.

In the context of the present invention ribozyles comprise, inter alia, hammerhead ribozymes, hammerhead ribozymes with altered core sequences or deoxyribozymes (see, e.g., Santoro, Proc. Natl. Acad. Sci. USA 94 (1997), 4262) and may comprise natural and in vitro selected and/or synthesized ribozymes. Nucleic acid molecules according to the present invention which are complementary to nucleic acid molecules coding for proteins/(poly)peptides regulating, causing or contributing to obesity and/or encoding a mammalian (poly)peptide involved in the regulation of body weight (see herein below) may be used for the construction of appropriate ribozymes (see, e.g., EP-B1 0 291 533, EP-A10 321 201, EP-A2 0 360 257) which specifically cleave nucleic acid molecules of the invention. Selection of the appropriate target sites and corresponding ribozymes can be done as described for example in Steinecke, Ribozymes, Methods in Cell Biology 50, Galbraith, eds. Academic Press, Inc. (1995), 449-460.

Furthermore, modulation of SOUP1 expression may be effected by using RNA interference, e.g. using short double-stranded RNA molecules, or precursors thereof or DNA molecules encoding such RNA molecules (see e.g. WO 02/44321).

The present invention also relates to a host cell transfected or transformed with the vector of the invention or a non-human host carrying the vector of the present invention, i.e. to a host cell or host which is genetically modified with a nucleic acid molecule according to the invention or with a vector comprising such a nucleic acid molecule. The term “genetically modified” means that the host cell or host comprises in addition to its natural genome a nucleic acid molecule or vector according to the invention which was introduced into the cell or host or into one of its predecessors/parents. The nucleic acid molecule or vector may be present in the genetically modified host cell or host either as an independent molecule outside the genome, preferably as a molecule which is capable of replication, or it may be stably integrated into the genome of the host cell or host.

The host cell of the present invention may be any prokaryotic or eukaryotic cell. Suitable prokaryotic cells are those generally used for cloning like E. coli or Bacillus subtilis. Furthermore, eukaryotic cells comprise, for example, fungal or animal cells. Examples for suitable fungal cells are yeast cells, preferably those of the genus Saccharomyces and most preferably those of the species Saccharomyces cerevisiae. Suitable animal cells are, for instance, insect cells, vertebrate cells, preferably mammalian cells, such as e.g. CHO, Hela, NIH3T3, MOLT-4, Jurkat, K562, HepG2, 3T3-442A, 3T3-L1 (and derivatives thereof), HIB-1B (see Villena, Biochem J. 331 (1998), 121-127), HEK 293, PAZ6 (see, Strobel, Diabetologia 42 (1999), 527-533). Further suitable cell lines known in the art are obtainable from cell line depositories, like the American Type Culture Collection (ATCC).

In a more preferred embodiment the host cell which is transformed with the vector of the invention is a mammalian cell, particularly an adipose cell, a brain cell, a hepatic cell, an epithelial cell, a blood cell or a cell (line) derived therefrom.

Non-human hosts are preferably non-human mammals, most preferably mice, rats, sheep, calves, dogs, monkeys or apes and may comprise Psammomis obesus. Said mammals may be indispensable for developing a cure, preferably a cure for obesity, adipositas, eating disorders and/or disorders leading to a pathological body mass/body weight. Furthermore, the hosts of the present invention may be partially useful in producing the (poly)peptides (or fragments thereof) of the invention. It is envisaged that said (poly)peptide (or fragments thereof) be isolated from said host.

The non-human host of the present invention may also be a non-human transgenic animal as described herein below (see Example 10). Particularly, the present invention envisages non-human transgenic animals comprising a mutated form of the nucleic acid molecules of the invention or non-human transgenic animals wherein the nucleic acid molecule of the present invention has been deleted and/or inactivated. Said deletion may be a partial deletion. Particularly preferred non-human transgenic animals are Drosophila, Nematodes (like C. elegans), mice, rat, sheep and the like.

Furthermore, the present invention relates to a method of producing a (poly)peptide encoded by the nucleic acid molecule of the invention comprising culturing the host cell of the present invention under suitable conditions that allow the synthesis of said (poly)peptide and recovering and/or isolating the (poly)peptide produced from the culture.

The transformed host cells can be grown in fermentors and cultured according to techniques known in the art to achieve optimal cell growth. The (poly)peptide of the invention can then be isolated from the growth medium, cellular lysates, cellular membrane fractions or inclusion bodies. Once expressed, the protein of the present invention can be purified according to standard procedures of the art, including ammonium sulfate precipitation, affinity columns, column chromatography, gel electrophoressis and the like; see, Scopes, “Protein Purification”, Springer-Verlag, N.Y. (1982). For example, “Current Protocols in Molecular Biology” (2000, John Wiley and Sons) provide for purification protocols. Further purification schemes are known in the art and provide also for the purification of membrane proteins. For example purification from expression in yeast/yeast expression systems is described in Murdza-Inglis (1991), JBC 266, 11871-11875, purification/expression in bacteria has been disclosed in Kaplan (1996), J. Bioenerg. Biomembr. 28, 41-47 or in eukaryotic cells (Casteilla (1990), PNAS 87, 5124-5128). Substantially pure proteins of at least about 60%, at least about 70%, at least about 80% or at least about 90 to 97% homogeneity are preferred, and 98 to 99% or more homogeneity are most preferred, for pharmaceutical uses. Once purified, partially or to homogeneity as desired, the proteins may then be used therapeutically (including extracorporeally) or in developing and performing assay procedures.

Additionally, the present invention relates to a (poly)peptide encoded by the nucleic acid molecule of the invention or produced by or obtainable by the above-described method. The term “(poly)peptide” as employed herein denotes either a peptide, a full-length protein or (a) fragment(s) thereof. A peptide is preferably a fragment of the (poly)peptide of the invention. The term “(poly)peptide comprises (a) peptide(s) or (a) (poly)peptide(s) which encompass amino acid chains of any length, wherein the amino acid residues are linked by covalent peptide bonds. Preferably, said amino acid chains of a “peptide” comprise at least 10 amino acids, more preferably at least 20, more preferably at least 30, more preferably at least 40, even more preferably at least 50 and, most preferably at least 60 amino acids. It is even more preferred that the (poly)peptides of the invention comprise at least 100, more preferred at least 200, more preferred at least 300, more preferred at least 400, more preferred at least 500, even more preferred at least 600 amino acids.

The term “or (a) fragment(s) thereof” as employed in the present invention and in context with (poly)peptides of the invention, comprises specific peptides, amino acid stretches of the (poly)peptides as disclosed herein. It is preferred that said “fragment(s) thereof” is/are functional fragment(s). The term “functional fragment” denotes a part of the above identified (poly)peptide of the invention which fulfils, at least in part, physiological and/or structural activities of the (poly)peptide of the invention. It is, however, also envisaged that said fragment functions as intervening and/or inhibiting molecule for the (poly)peptide of the invention. For example, it is envisaged that fragments of the (poly)peptide of the invention may structurally and/or physiologically interact with the (poly)peptide of the invention and thereby inhibit the function of said (poly)peptide.

The (poly)peptides of the present invention may be recombinant (poly)peptides expressed in host cells like bacteria, yeasts, or other eukaryotic cells, like mammalian or insect cells. Alternatively, they may be isolated from viral preparations. In another embodiment of the present invention, synthetic (poly)peptides may be used. Therefore, such a (poly)peptide may be a (poly)peptide as encoded by the nucleic acid molecule of the invention which only comprises naturally occurring amino acid residues, but it may also be a (poly)peptide containing modifications. These include covalent derivatives, such as aliphatic esters or amides of a carboxyl group, O-acetyl derivatives of hydroxyl containing residues and N-acyl derivatives of amino group containing residues. Such derivatives can be prepared by linkage to reactable groups which are present in the side chains of amino acid residues and at the N- and C-terminus of the protein. Furthermore, the (poly)peptide can be radiolabeled or labeled with a detectable group, such as a covalently bound rare earth chelate, or conjugated to a fluorescent moiety. The (poly)peptide of the present invention can be, for example, the product of expression of a nucleotide sequence encoding such a (poly)peptide, a product of chemical modification or can be purified from natural sources, for example, viral preparations. Furthermore, it can be the product of covalent linkage of (poly)peptide domains.

The peptides/(poly)peptides may also be produced by biochemical or synthetic techniques. Those methods are known to those of ordinary skill in the art (see, e.g. Merrifield, J. Am. Chem. Soc. 85 (1963), 2149-2146; Stewart, “Solid Phase Peptide Synthesis”, WH Freeman Co, San Francisco (1969); Scopes, “Protein Purification”, Springer Verlag, New York, Heidelberg, Berlin (1987); Janson, “Protein Purification, Principles, High Resolution Methods and Applications”, VCH Publishers, New York, Weinheim, Cambridge (1989); Wrede, “Concepts in Protein Engineering and Design”, Walter de Gruyter, Berlin, New York (1994)).

Additionally, within the scope of the invention are peptides/(poly)peptides wherein the above mentioned amino acid(s) and/or peptide bonds have been replaced by functional analogs, inter alia by peptidomimetics. Peptidomimetics is well known in the art and corresponding art describing this method are mentioned below. Therefore, the present invention also encompasses functional derivatives and/or analogues of said peptides comprising a specific SOUP1-derived peptide. Further methods for the preparation of peptides/(poly)peptides are described in Sambrook et al., loc. cit., or in Oxender and Fox (1987) “Protein Engineering”, Alan Liss Inc. New York. Protein preparation of chemical derivates and/or analogues are described in, for example, Beilstein “Handbook of Organic Chemistry”, Springer Edition New York, or in “Organic Synthesis”, Wiley, New York.

The present invention also relates to a fusion protein comprising the (poly)peptide of the invention or (a) fragment thereof. Therefore, in addition to the (poly)peptides of the present invention, said fusion protein can comprise at least one further domain, said domain being linked by covalent or non-covalent bonds. The linkage can be based on genetic fusion according to the methods known in the art (Sambrook et al., loc. cit., Ausubel, “Current Protocols in Molecular Biology”, Green Publishing Associates and Wiley Interscience, N.Y. (1989)) or can be performed by, e.g., chemical cross-linking as described in, e.g., WO 94/04686. The additional domain present in the fusion protein comprising the (poly)peptide of the invention may preferably be linked by a flexible linker, advantageously a (poly)peptide linker, wherein said (poly)peptide linker preferably comprises plural, hydrophilic, peptide-bonded amino acids of a length sufficient to span the distance between the C-terminal end of said further domain and the N-terminal end of the peptide, (poly)peptide or antibody or vice versa. The above described fusion protein may further comprise a cleavable linker or cleavage site, which, for example, is specifically recognized and cleaved by proteinases or chemical agents. Additionally, said at least one further domain may be of a predefined specificity or function. In this context, it is understood that the (poly)peptides of the invention may be further modified by conventional methods known in the art. This allows for the construction of fusion proteins comprising the (poly)peptide of the invention and other functional amino acid sequences, e.g., organelle localization signals, transactivating domains, DNA-binding domains, hormone-binding domains, protein tags (e.g. GST, GFP, h-myc peptide, FLAG, HA peptide, Strep), transmembrane domains or fatty acid attachment motifs which may be derived from heterologous proteins.

The fusion protein of the invention may also be a mosaic (poly)peptide comprising at least two epitopes of the (poly)peptide of the invention wherein said mosaic (poly)peptide lacks amino acids normally intervening between the epitopes in the native SOUP1 protein.

Inter alia, such mosaic (poly)peptides are useful in the applications and methods described herein, since they may comprise within a single peptide or (poly)peptide a number of relevant epitopes possibly presented linearly or as multi-antigen peptide system in a case of lysines. Relevant epitopes can be separated by spacer regions.

It is in particular preferred that the fusion protein of the invention comprising said polypeptide or (a) fragment(s) thereof comprise(s) at least one, preferably at least two, more preferably at least three, more preferably at least four, more preferably at least five and most preferably six amino acid sequences as depicted in any one of SEQ ID NOs: 15 to 50, 61 or 62. As disclosed herein above, said sequence relate to specifically deduced transmembrane regions of the SOUP1 proteins described herein. It is particularly preferred that the fusion protein of the invention comprises at least one and most preferably at least six amino acid sequences as depicted in any one of SEQ ID NOs: 27 to 50. It is also envisaged that said fusion protein comprises transmembrane regions which are derived from different species, e.g. from human, mouse or zebrafish. Yet, most preferred are fusion proteins which comprise transmembrane regions from one species.

The nucleic acid molecule, the (poly)peptide (as well as the antibody or fragment or derivative thereof, the aptamer or other receptor described herein), the fusion protein, the mosaic (poly)peptide or the anti-sense oligonucleotide of the invention may be detectably labeled. A variety of techniques are available for labeling biomolecules, are well known to the person skilled in the art and are considered to be within the scope of the present invention. Such techniques are, e.g., described in Tijssen, “Practice and theory of enzyme immuno assays”, Burden, R H and von Knippenburg (Eds), Volume 15 (1985), “Basic methods in molecular biology”; Davis L G, Dibmer M D; Battey Elsevier (1990), Mayer et al., (Eds) “Immunochemical methods in cell and molecular biology” Academic Press, London (1987), or in the series “Methods in Enzymology”, Academic Press, Inc.

There are many different labels and methods of labeling known to those of ordinary skill in the art. Examples of the types of labels which can be used in the present invention include enzymes, radioisotopes (like ³²P or ¹²⁵I), colloidal metals, fluorescent compounds/fluorochromes (like fluorescein, rhodamine, Texas Red, etc.), chemiluminescent compounds, and chemi- or bioluminescent compounds (like dioxetanes, luminol or acridiniums).

Commonly used labels furthermore comprise, inter alia, enzymes (like horse radish peroxidase, β-galactosidase, alkaline phosphatase), biotin or digoxygenin. Labeling procedures, like covalent coupling of enzymes or biotinyl groups, iodinations, phosphorylations, biotinylations, random priming, nick-translations, tailing (using terminal transferases) are well known in the art.

Detection methods comprise, but are not limited to, autoradiography, fluorescence microscopy, direct and indirect enzymatic reactions, etc.

The present invention furthermore additionally relates to an antibody or a fragment or derivative thereof or an antiserum or an aptamer or another receptor specifically recognizing an epitope on the nucleic acid, or the (poly)peptide of the invention. The general methodology for producing antibodies is well-known and has, for monoclonal antibodies, been described in, for example, Köhler and Milstein, Nature 256 (1975), 494 and reviewed in J. G. R. Hurrel, ed., “Monoclonal Hybridoma Antibodies: Techniques and Applications”, CRC Press Inc., Boco Raron, Fla. (1982). In accordance with the present invention the term “antibody” relates to monoclonal or polyclonal antibodies. Polyclonal antibodies (antiserum) can be obtained according to conventional protocols. Antibody fragments or derivatives comprise F(ab′)₂, Fab, Fv or scFv fragments: see, for example, Harlow and Lane, “Antibodies, A Laboratory Manual”, CSH Press 1988, Cold Spring Harbor, N.Y. Preferably the antibody of the invention is a monoclonal antibody. Furthermore, in accordance with the present invention, the derivatives of the invention can be produced by peptidomimetics. In the context of the present invention, the term “aptamer” comprises nucleic acids such as RNA, ssDNA (ss=single stranded), modified RNA, modified ssDNA or PNAs which bind a plurality of target sequences having a high specificity and affinity. Aptamers are well known in the art and, inter alia, described in Famulok, Curr. Op. Chem. Biol. 2 (1998), 320-327. The preparation of aptamers is well known in the art and may involve, inter alia, the use of combinatorial RNA libraries to identify binding sites (Gold, Ann. Rev. Biochem. 64 (1995), 763-797). Said other receptors may, for example, be derived from said antibody etc. by peptidomimetics. The specificity of the recognition implies that other known proteins, molecules are not bound. A suitable host for assessing the specificity would imply contacting the above recited compound comprising an epitope of the nucleic acid molecule or the (poly)peptide of the invention as well as corresponding compounds e.g. from protein or nucleic acid molecules known in the art, for example in an ELISA format and identifying those antibodies etc. that only bind to the compound of the invention but do not or to no significant extent cross-react with said corresponding compounds.

The invention also relates to an anti-sense oligonucleotide of a nucleic acid molecule of the invention. As said anti-sense oligonucleotide may be employed in scientific as well as in diagnostic or in therapeutic purposes.

The invention furthermore provides for a non-human animal expressing the polypeptide of the invention or the fusion protein of the invention or which is transfected with the vector of the invention which comprises the nucleic acid molecule of the invention.

It is envisaged, for example, that the non-human animal over- or under-expresses the polypeptide of the invention. Furthermore, the invention relates to a non-human animal, wherein the nucleic acid molecule of the invention or a homolog, paralog or ortholog thereof is silenced and/or mutated.

The above mentioned non-human animal is preferably selected from the group consisting of mouse, rat, sheep, hamster, pig, dog, monkey, rabbit, calf, horse, nematodes, fly and fish.

The invention also relates to transgenic non-human animals such as transgenic mice, rats, hamsters, dogs, monkeys, rabbits, pigs, C. elegans, Drosophila, fish (like zebrafish or torpedofish) comprising a nucleic acid molecule or vector of the invention. Said animal may have one or several copies of the same or different nucleic acid molecules encoding one or several forms of the (poly)peptide of the invention, regulating, causing or contributing to obesity or involved in the regulation of body weight. These animals are partially useful as research models for obesity, adipositas, eating disorders, wasting and/or other disorders of body weight/body mass as described herein. Furthermore, said transgenic non-human animals are well suited for, e.g., pharmacological studies of drugs in connection with mutant forms of the above described SOUP1 protein.

In another embodiment, the present invention relates to the use of the nucleic acid molecule, the vector, the host, the polypeptide, the fusion protein, the antibody, fragment or derivative thereof or an aptamer or another receptor or the anti-sense oligonucleotide of the invention for controlling the function of a gene and/or a gene product which is influenced and/or modified by a polypeptide as defined herein, e.g. SOUP1. Said influence/modification may occur by direct interaction between proteins/protein fragments and/or by providing metabolic compounds, or ions that are necessary for the function, activity and/or expression of said gene and/or gene product. It is particularly preferred that said gene and/or gene product is a gene and/or gene product expressed in organelles. Said organelle may be, inter alia, a mitochondrium or a peroxisome.

It is particularly preferred that said gene and/or gene product is a member of the UCP family. Members of the UCP family are well known and described herein above.

The present invention furthermore provides for a composition comprising the nucleic acid molecule, the vector, the host, the polypeptide, the fusion protein, the antibody, fragment or derivative thereof or an aptamer or another receptor or the anti-sense oligonucleotide of the invention. Said composition may be, inter alia, a diagnostic composition or a pharmaceutical e.g. therapeutic composition for use e.g in human or veterinary medicine. Further, the composition may comprise suitable carriers, diluents and/or adjuvants.

In addition, the present invention provides for the use of the composition as defined herein for detecting and/or verifying an disorder in cells, cell masses, organs and/or subjects and/or for the treatment, alleviation and/or prevention of an disorder in cells, cell masses, organs and/or subjects. Said disorder may be a metabolic disorder or a mitochondrial disorder, whereby mitochondrial disorders comprise disorders like deafness, retinopathies, progressive enzelopathies, ataxias, spastic paraplegia, metabolic acidosis and others.

Said metabolic disorder may comprise obesity, adipositas, eating disorders (bulimia nervosa, anorexia nervosa), cachexia (wasting), pancreatic dysfunction (like diabetes, in particular type 2 diabetes) and/or a disorder related to ROS (reactive oxygen species) production (in particular responses to infections, in aging and cancerogenesis).

For example, it has been shown that UCPs are involved in pancreatic disorders, e.g. diabetes. A role for uncoupling proteins in diabetes was demonstrated by induction of UCP3 in Streptozotocin-induced diabetes in rodents (see, inter alia, Hidaka, Proc Soc Exp Biol Med 224: 172-177 (2000), Hidaka, Diabetes 48: 430-435 (1999)).

Furthermore it was shown that UCP2 expression in pancreatic beta-cells influences beta-cell function and insulin secretion (Wang, Diabetes 48: 1020-1025 (1999); Chan, Diabetes 48: 1482-1486 (1999)).

Reactive oxygen species (ROS) can lead to membrane dysfunction, DNA damage and inactivation of proteins. Pathological consequences include cancer, arthritis and neurodegenarative disease. ROS limiting metabolism is a major mechanism to protection from cellular damage. In particular obesity can cause increased oxidative stress (Hayes, Free Radic Res 31: 273-300 (1999); Yang, Arch Biochem Biophys 378: 259-268 (2000)).

In contrast, increased ROS production in macrophages can improve immune response. So are UCP2 knockout mice more resistant against infection with certain pathogens.

Therefore, the compounds of the present invention, being capable of modifying, inter alia, UCPs may be well suited for the above identified purposes.

In a further embodiment, the present invention relates to the use of the nucleic acid molecule, the vector, the host, the polypeptide, the fusion protein, the antibody, fragment or derivative thereof or an aptamer or another receptor or the anti-sense oligonucleotide for identifying substances capable of interacting with the polypeptide as defined in herein. Said substance is capable of interacting with said polypeptide may be (an) antagonist(s) or (an) agonist(s).

In yet a further embodiment, the present invention provides for a method of identifying a polypeptide or (a) substance(s) involved in cellular metabolism in an animal or capable of modifying homeostasis comprising the steps of:

(a) testing a collection of polypeptides or substances for interaction with the polypeptide of the invention (a) fragment(s) thereof or the fusion protein of the invention or (a) fragment(s) thereof using a readout system; and

(b) identifying polypeptides or substances which test positive for interaction in step (a).

The term “cellular metabolism” as used herein above may comprise an metabolic event involved in the regulation of ion-, vitamin- or metabolite-transport across organelle membranes. These transport events or the regulation thereof may influence energy homeostasis, accumulation of storage compounds and/or radical production/elimination.

The polypeptide or substance identified by the method disclosed herein above may be, inter alia, a polypeptide or a substance interacting directly or indirectly (e.g. via linker proteins or via physiological parameters) with the polypeptide of the invention, i.e. with SOUP1 proteins and/or a fragment thereof. The term “substance” as used herein broadly relates to physiological and non-physiological, e.g. synthetic substances.

The term “fragment” as used in the specification relates to fragments of SOUP1 proteins which comprise at least 5, preferably at least 10, more preferably at least 15 and even more preferably at least 25 amino acids and/or further comprise at least one, preferably at least 2, more preferably at least 3, more preferably at least 4, more preferably at least 5 and most preferably at least 6 transmembrane regions. Yet, it is also envisaged that a fragment as used herein represents the N- or C-terminus of a SOUP1 protein.

Said testing for interaction of step (a) as described herein above may be carried out by methods known to the skilled artisan and were described herein. In particular these assays comprise biochemical, immunological and/or molecular biological assays.

Said interaction assays employing read-out systems are well known in the art and comprise, inter alia, two hybrid screenings (as, described, inter alia, in EP-0 963 376, WO 98/25947, WO 00/02911) GST-pull-down columns, co-precipitation assays from cell extracts as described, inter alia, in Kasus-Jacobi, Oncogene 19 (2000), 2052-2059, “interaction-trap” systems (as described, inter alia, in U.S. Pat. No. 6,004,746) expression cloning (e.g. lamda gtII), phage display (as described, inter alia, in U.S. Pat. No. 5,541,109), in vitro binding assays and the like. Further interaction assay methods and corresponding read out systems are, inter alia, described in U.S. Pat. No. 5,525,490, WO 99/51741, WO 00/17221, WO 00/14271 or WO 00/05410.

Similarly, interacting molecules/(poly)peptides may be deduced by cell-based techniques well known in the art. These assays comprise, inter alia, the expression of reporter gene constructs or “knock-in” assays, as described, for, e.g., the identification of drugs/small compounds influencing the gene expression. Said “knock-in” assays may comprise “knock-in” in tissue culture cells, as well as in (transgenic) animals. Examples for successful “knock-ins” are known in the art (see, inter alia, Tanaka, J. Neurobiol. 41 (1999), 524-539 or Monroe, Immunity 11 (1999), 201-212). Furthermore, biochemical assays may be employed which comprise, but are not limited to, binding of the (poly)peptides of the invention (or (a) fragment(s) thereof) to other molecules/(poly)peptides, peptides or binding of the (poly)peptides of the invention (or (a) fragment(s) thereof) to itself (themselves) (dimerizations, oligomerizations, multimerizations) and assaying said interactions by, inter alia, scintillation proximity assay (SPA) or homogenous time-resolved fluorescence assay (HTRFA).

Further method(s) which may be employed comprises FRET (fluorescence resonance energy transfer; as described, inter alia, in Ng, Science 283 (1999), 2085-2089), or fluorescence polarization assays. These methods are well known in the art and inter alia described in Fernandez, Curr. Opin. Chem. Biol. 2 (1998), 547-603.

Said “testing of interaction” may also comprise the measurement of a complex formation. The measurement of a complex formation is well known in the art and comprises, inter alia, heterogeneous and homogeneous assays. Homogeneous assays comprise assays wherein the binding partners remain in solution and comprise assays, like agglutination assays. Heterogeneous assays comprise assays like, inter alia, immuno assays, for example, ELISAs, RIAs, IRMAs. FIAs, CLIAs or ECLs.

Further methods and assays for identifying interaction and/or binding partners of the (poly)peptides of the invention or for the identification of agents/compounds which are capable of interfering with the binding of the (poly)peptides of the invention with this (specific) intracellular binding partners/targets are disclosed herein below. Said additional and/or further method(s) and assay(s) may also be employed in the above described method for identifying a (poly)peptide involved in the regulation of body weight and/or capable of interacting with the SOUP1 (poly)peptide of the invention.

Any measuring or detection step of the method(s) of the present invention may be assisted by computer technology. For example, in accordance with the present invention, said detection and/or measuring step can be automated by various means, including image analysis, spectroscopy or flow cytometry.

In yet another embodiment, the present invention relates to the method(s) described herein above, which further comprises the step of identifying the nucleic acid molecule(s) encoding the one or more interacting (poly)peptides.

The identification of such nucleic acid molecule(s) is well known in the art and comprises, inter alia, the use of specific and/or degenerate primers. Furthermore, recombinant technologies as described in Sambrook, loc. cit. or in Glick (1994), “Molecular Biotechnology”, ASM Press, Washington may be employed.

In yet a further embodiment, the present invention relates to a method of identifying a polypeptide or (a) substance(s) involved in cellular metabolism in an animal or capable of modifying homeostasis comprising the steps of

(a) testing a collection of polypeptides or substances for interaction with the polypeptide of the invention or identified by the method described herein above; and

(b) identifying polypeptides that test positive for interaction in step (a); and optionally

(c) repeating steps (a) and (b) with the polypeptides identified one or more times wherein the newly identified polypeptide replaces the previously identified polypeptide as a bait for the identification of a further interacting polypeptide.

The methods described herein above may further comprise the step of identifying the nucleic acid molecule(s) encoding the one or more interacting (poly)peptides.

The present invention also provides for the use of nucleic acid molecules as described herein or of polypeptides as described herein for the detection and/or isolation of genes and/or gene products involved in functional cascades of cell metabolism, in particular of energy metabolism.

Additionally, the present invention relates to a method of identifying a polypeptide involved in the regulation of body weight in a mammal comprising the steps of

(a) contacting a collection of (poly)peptides with the polypeptide of the invention or (a) fragment(s) thereof or the fusion protein of the invention or (a) fragment(s) thereof under conditions that allow binding of said (poly)peptides;

(b) removing (poly)peptides from said collection of (poly)peptides that did not bind to said polypeptide of the invention or the fusion protein of the invention in step (a); and

(c) identifying (poly)peptides that bind to said polypeptide of the invention or the fusion protein of the invention.

The method as described herein above may be carried out by the person skilled in the art without further ado. Said “contacting” of step (a) may, inter alia, be carried out in solution employing (magnetic) beads coupled with the (poly)peptide of the invention and/or fragments thereof. Non-bound (poly)peptides may be easily removed by methods known in the art, comprising, for example, magnetic separation, gravity, affinity column systems and corresponding washes and the like.

Methods for identifying bound (poly)peptides are well known in the art and comprise, inter alia, SDS PAGE analysis and Western blotting. Furthermore, techniques like 2D-gel electrophoresis, in-gel digests, microsequencing, N-terminal sequencing, MALDI-MS, analysis of peptides in mass spectroscopy, peptide mass fingerprinting, PSD-MALDI-MS and/or (micro-) HPLC. Separated polypeptdies to be identified may be further analyzed by, inter alia, Edman-degradation, MALDI-MS methods, ladder sequencing (Thiede, FEBS 357 (1995), 65).

By use of the above described and mentioned methods (and others known in the art) amino acid sequences of the (poly)peptides to be identified can be deduced and sequenced. From these sequenced amino acid fragments, degenerative oligonucleotides may be deduced and synthesized that may be used to screen, for example, genomic or cDNA libraries to identify and clone the corresponding gene/cDNA.

Furthermore, phage display approaches may be employed in the method(s) of this invention. Phage display allows the identification of proteins that interact with a molecule of interest. Libraries of phage, each displaying a different peptide epitope are tested for binding to the molecule of interest. Bound phages can be purified and the insert encoding the peptide epitope may be sequenced. Phage display kit(s) are known in the art and commercially available, e.g., Display Systems Biotech Cat. No. 300-110.

The present invention relates, in yet another embodiment to the method(s) described herein, wherein said (poly)peptide of the invention is fixed to a solid support.

Solid supports are well known in the art and comprise, inter alia, commercially available column materials, polystyrene beads, latex beads, magnetic beads, colloid metal particles, glass and/or silicon chips and surfaces, nitrocellulase strips, membranes, sheets, duracytes, wells and walls of reaction trays, plastic tubes etc. Suitable methods for fixing/immobilizing said (poly)peptide(s) of the invention are well known and include, but are not limited to ionic, hydrophobic, covalent interactions and the like.

In a particular preferred embodiment, said solid support is a gel filtration or an affinity chromatography material.

In a yet more preferred embodiment of the method of the invention as described herein above, said binding (poly)peptides are released prior to said identification in step (c).

Said release may be effected by elution. Such elution methods are well known in the art and comprise, inter alia, elution with solutions of different ionic strength or different pH, or with intercalating or competing agents/molecules/peptides.

Furthermore, in a yet more preferred embodiment, the present invention relates to the above described method of the invention, wherein said method further comprises the step of identifying the nucleic acid molecule(s) encoding the one or more binding (poly)peptides.

As pointed out herein above, said nucleic acid molecule(s) may be deduced, inter alia, by employing degenerate primers/oligonucleotides in order to detect the corresponding gene(s) and/or cDNA(s) or by expression cloning.

A method of identifying a compound influencing the expression of the nucleic acid molecule of the invention comprising the steps of

(a) contacting a host carrying an expression vector comprising the nucleic acid molecule of the invention or the nucleic acid molecule identified by the method of the invention operatively linked to a readout system with a compound or a collection of compounds;

(b) assaying whether said contacting results in a change of signal intensity provided by said readout system; and, optionally,

(c) identifying a compound within said collection of compounds that induces a change of signal in step (b);

wherein said change in signal intensity correlates with a change of expression of said nucleic acid molecule.

Furthermore, the present invention provides for a method of identifying a compound influencing the activity of a (poly)peptide as defined herein above comprising the steps of

(a) contacting a host carrying an expression vector comprising the nucleic acid molecule of the invention operatively linked to a readout system and/or carrying a (poly)peptide of the invention linked to a readout system with a compound or a collection of compounds;

(b) assaying whether said contacting results in a change of signal intensity provided by said readout system; and, optionally

(c) identifying a compound within said collection of compounds that induces a change of signal in step (b);

wherein said change in signal correlates with a change in activity of said (poly)peptide.

The term “activity” as used herein above in context of the method of the invention also comprises the “function” of (a) (poly)peptide(s) of the invention. Said function may comprise, as mentioned herein above, enzymatic activities or other functions, like, inter alia, involvement in signalling pathways. Such activities and modulators of such activities may be determined and/or identified by convenient in vitro or in vivo assays as described herein or by variations thereof. The underlying technology is widely and commonly known to the person skilled in the art.

Readout systems operatively linked to the nucleic acid molecules of the invention or linked to the (poly)peptides of the invention are disclosed herein and comprise, but are not limited to, assays based on radioactive lables, luminescence, fluorescence, etc. Inter alia, said readout system may comprise fluorescence resonance energy transfer (FRET). The above described methods are particularly useful in (automated) high-throughput screenings. In context of this invention, the above mentioned “readout system opertatively linked to the nucleic acid molecules of the invention” also comprises readout systems which are located on different molecules. e.g. nucleic acid molecules, like, inter alia, other plasmids, vectors etc.

Said host of step (a) of the methods described herein above may be a eukaryotic host cell. Said host cell may be a yeast cell or a plant cell. It is particularly preferred that said eukaroytic host cell is a mammalian host cell. However, said host cell may also be a prokaryotic cell, e.g. a bacterium. Particularly preferred are prokaryotic (host) cells as described herein above.

The term “compound” in the method(s) of the invention includes a single substance or a plurality of substances which may or may not be identical. Said compound(s) may be comprised in, for example, samples, e.g., cell extracts from, e.g., plants, animals or microorganisms. Furthermore, said compound(s) may be known in the art but hitherto not known to be capable of influencing the activity of (a) (poly)peptide(s) of the invention or not known to be capable of influencing the expression of the nucleic acid molecule of the invention, respectively. The plurality of compounds may be, e.g., added to a sample in vitro, to the culture medium or injected into the cell.

If a sample (collection of compounds) containing (a) compound(s) is identified in the method(s) of the invention, then it is either possible to isolate the compound from the original sample identified as containing the compound in question or one can further subdivide the original sample, for example, if it consists of a plurality of different compounds, so as to reduce the number of different substances per sample and repeat the method with the subdivisions of the original sample. It can then be determined whether said sample or compound displays the desired properties by methods known in the art such as described herein. Depending on the complexity of the samples, the steps described above can be performed several times, preferably until the sample identified according to the method of the invention only comprises a limited number of or only one substance(s). Preferably said sample comprises substances of similar chemical and/or physical properties, and most preferably said substances are identical. The methods of the present invention can be easily performed and designed by the person skilled in the art, for example in accordance with other cell based assays described in the prior art (see, e.g., EP-A-0 403 506). Furthermore, the person skilled in the art will readily recognize which further compounds and/or cells may be used in order to perform the methods of the invention, for example, host cells as described herein above or enzymes, if necessary, that, e.g., convert a precursor compound into the active compound which in turn influences the expression of the nucleic acid molecule of the invention and/or influences the activity of (a) (poly)peptide of the invention. Such adaptation of the method of the invention is well within the skill of the person skilled in the art and can be performed without undue experimentation.

Compounds which can be used in accordance with the method of the present invention include, inter alia, peptides, proteins, nucleic acids including cDNA expression libraries, antibodies, small organic compounds, ligands, PNAs and the like. Said compounds can also be functional derivatives or analogues of known activators or inhibitors. Methods for the preparation of chemical derivatives and analogues are well known to those skilled in the art and are described in, for example, Beilstein, loc. cit. Furthermore, said derivatives and analogues can be tested for their effects according to methods known in the art and/or as described herein. Furthermore, peptidomimetics and/or computer aided design of appropriate activators or inhibitors of the expression of the nucleic acid molecules of the invention or of the activity of (a) (poly)peptide of the invention can be used, for example, according to the methods described herein. Appropriate computer systems for the computer aided design of, e.g., proteins and peptides are described in the prior art, for example, in Berry, Biochem. Soc. Trans. 22 (1994), 1033-1036; Wodak, Ann. N.Y. Acad. Sci, 501 (1987), 1-13; Pabo, Biochemistry 25 (1986), 5987-5991. The results obtained from the above-described computer analysis can be used in combination with the method of the invention for, e.g., optimizing known compounds, substances or molecules. Appropriate compounds can also be identified by the synthesis of peptidomimetic combinatorial libraries through successive chemical modification and testing the resulting compounds, e.g., according to the methods described herein. Methods for the generation and use of peptidomimetic combinatorial libraries are described in the prior art, for example in Ostresh, Methods in Enzymology 267 (1996), 220-234 and Dorner, Bioorg. Med. Chem. 4 (1996), 709-715. Furthermore, the three-dimensional and/or crystallographic structure of inhibitors or activators of SOUP1 protein or the soup1 nucleic acid molecule can be used for the design of peptidomimetic inhibitors or activators of the (poly)peptide of the invention to be tested in the method of the invention (Rose, Biochemistry 35 (1996), 12933-12944; Rutenbern Bioorg. Med. Chem. 4 (1996), 1545-1558).

In a particularly preferred embodiment, the above described methods of the invention are method(s) wherein said change in signal intensity is an increase in signal intensity or a decrease in signal intensity. The above described method(s) of the invention for identifying compounds influencing the expression of the nucleic acid molecule of the invention and/or the activity of the (poly)peptide of the invention may also be employed for screening of said compound(s).

In yet a further embodiment, the invention provides for a method of assessing the impact of the expression of one or more polypeptides or of one or more fusion proteins of the invention in an animal comprising the steps of

(a) overexpressing a nucleic acid molecule coding for a polypeptide or a fusion protein of the invention in said animal; and

(b) determining whether the weight of said animal has increased, decreased, whether metabolic changes are induced and/or whether the eating behaviour is modified.

Similarly, the present invention also relates to a method of assessing the impact of the expression of one or more (poly)peptides or of one or more fusion proteins of the invention in an animal comprising the steps of

(a) underexpressing a nucleic acid molecule coding for a polypeptide or a fusion protein of the invention in said animal; and

(b) determining whether the weight of said animal has increased or decreased, whether metabolic changes are induced and/or whether the eating behaviour is modified.

Transgenic animals as described herein above may be particularly useful for the above described methods of assessing the impact of the expression of one or more (poly)peptide of the invention. The above mentioned “underexpression” of the nucleic acid molecule of the invention comprises, inter alia, full deletions of both alleles, or the deletion of any one allele. Furthermore, said term comprises the generation of a mutation which leads to the expression of a less functional protein/(poly)peptide in the test animal.

A method of screening for an agent which modulates the interaction of a polypeptide as defined herein above with a binding target/agent, comprising the steps of

(a) incubating a mixture comprising

(aa) a polypeptide of the invention, or a fragment thereof or a fusion protein of the invention or a fragment thereof;

(ab) a binding target/agent of said (poly)peptide or fusion protein or fragment thereof; and

(ac) a candidate agent

under conditions whereby said (poly)peptide, fusion protein or fragment thereof specifically binds to said binding target/agent at a reference affinity;

(b) detecting the binding affinity of said (poly)peptide, fusion protein or fragment thereof to said binding target to determine an (candidate) agent-biased affinity; and

(c) determining a difference between (candidate) agent-biased affinity and the reference affinity.

As pointed out herein above, a specific binding target/agent of the (poly)peptide(s) of the present invention may comprise molecules involved in signalling pathways and/or specific receptors contacting of the (poly)peptide of the invention. However, it is also envisaged that said binding target/agent of the (poly)peptide of the invention is said (poly)peptide itself, leading, inter alia, to dimerizations or multimerizations. Further (natural and artificial) binding targets/agents may be identified by methods known in the art and disclosed herein.

The “reference affinity” of the interaction of the (poly)peptides of the invention and its binding targets/agents may be established and/or deduced by methods known in the art. Said methods comprise, but are not limited to, in vitro and in vivo methods and may involve binding assays as described herein. In particular, said binding assays encompass any assay where the molecular interaction of the (poly)peptides of the invention with binding targets/agents be evaluated. Said binding target/agent may comprise natural (e.g. intracellular) binding targets/agents, such as, e.g., SOUP1-substrate, SOUP1 (poly)peptide itself, SOUP1 (poly)peptide regulators and/or molecules of signalling cascades. Within the scope of this invention are, however also non-natural binding partners of the (poly)peptide of the invention, which may comprise, e.g., antibodies or derivatives and/or fragments thereof, aptamers, as well as non-natural receptor molecules. Said binding targets/agents also comprise antagonists as well as agonists of the (poly)peptides of the present invention.

Specific affinities, activities and/or function of the (poly)peptide(s) of the invention may be determined by convenient in vitro, cell-based or in vivo assays, e.g. in vitro binding assays, cell culture assays, in animals (e.g. gene therapy, transgenics), etc. Binding assays encompass any assay where the molecular interaction of a (poly)peptide of the invention with a binding target is evaluated. The binding target may be a natural intracellular binding target such as oligomerization (dimerization, multimerization) of said (poly)peptide of the invention itself, a substrate or a regulating protein of said (poly)peptide of the invention or another regulator that directly modulates the activity or the (cellular) localization of the (poly)peptides of the invention. Further binding targets/agents comprise non-natural binding targets like (a) specific immune protein(s) such as an antibody, or an SOUP1 (poly)peptide specific agent such as those identified in screening assays as described below.

Specific screening assays are, inter alia, disclosed in U.S. Pat. No. 5,854,003 or in U.S. Pat. No. 5,639,858. Specific binding agents of the (poly)peptides of the invention may include SOUP1-specific receptors, such as those of the family of heptahelical receptors. Other natural SOUP1 binding targets are readily identified by screening cells, membranes and cellular extracts and fractions with the disclosed materials and methods and by other methods known in the art. For example, natural intracellular binding targets of the (poly)peptide of the invention may be identified with assays such as one-, two-, and three-hybrid screens. In addition, biochemical purification procedures, co-precipitation assays from cell extracts, “interaction-trap” systems, expression cloning (e.g. in bacteria using lambda gt11 or in eukaryotic cell systems using plasmid expression vectors), phage display, and the like, may be utilised for identification of natural soup1 binding agents. Non-natural intracellular binding agents may be obtained in screens of chemical libraries such as described below, etc.

The invention provides efficient methods of identifying pharmacological agents, compounds or lead compounds for agents active at the level of SOUP1 modulatable cellular function. Generally, these screening methods involve assaying for compounds, which modulate interaction of the (poly)peptides of the invention with a natural SOUP1 binding target. A wide variety of assays for binding agents are provided including labeled in vitro protein-protein binding assays, immunoassays, cell based assays, etc. The methods are amenable to automated, cost-effective high-throughput screening of chemical libraries for lead compounds and have immediate application in a broad range of domestic and international pharmaceutical and biotechnology drug development programs. Identified reagents find use in the pharmaceutical industries for animal and human trials; for example, the reagents may be derivatised and rescreened in vitro and in vivo assays to optimise activity and minimise toxicity for pharmaceutical development.

In vitro binding assays employ a mixture of components including a (poly)peptide of the invention, which may be part of a fusion product with another peptide or (poly)peptide(s), e.g. a tag for detection or anchoring, etc. The (poly)peptides of the invention or fragment(s) thereof used in the methods are usually added in an isolated, partially pure or pure form and are typically recombinantly produced. The assay mixture also comprises a candidate pharmacological agent at different concentrations. Candidate agents encompass numerous chemical classes, though typically they are organic compounds; preferably small organic compounds. Small organic compounds have a molecular weight of more than 50 Da yet less than about 2,500 Da, preferably less than about 1,000 Da, more preferably, less than about 500 Da. Candidate agents comprise functional chemical groups necessary for structural interactions with proteins and/or DNA, and typically include at least an amine, carbonyl, hydroxyl or carboxyl group, preferably at least two of the functional chemical groups, more preferably at least three. The candidate agents often comprise cyclical carbon or heterocyclic structures and/or aromatic or polyaromatic structures substituted with one or more of the aforementioned functional groups. Candidate agents are also found among biomolecules including peptides, saccharides, fatty acids, steroids, purine, pyrimidies, derivatives, structural analogues or combinations thereof and the like. Where the agent is or is encoded by a transfected nucleic acid, said nucleic acid is typically DNA or RNA.

Candidate agents are obtained from a wide variety of sources including libraries of synthetic or natural compounds. For example, numerous means are available for random and directed synthesis of a wide variety of organic compounds and biomolecules. Alternatively, libraries of natural compounds in the form of bacterial, fungal, plant, and animal extracts are available or readily produced. Additionally, natural and synthetically produced libraries and compounds are readily modified through conventional chemical, physical, and biochemical means. In addition, known pharmacological agents may be subject to directed or random chemical modifications, such as acylation, alkylation, esterification, amidification, etc., to produce structural analogues.

A variety of other reagents may also be included in the mixture. These include reagents required as biochemical energy sources, e.g. ATP or ATP analogues, nucleic acids, e.g. in nucleic acids binding assays, salts, buffers, neutral proteins, e.g. albumin, detergents, etc., which may be used to facilitate optimal protein-protein and/or protein-nucleic acid binding and/or reduce non-specific or background interactions, etc. Also, reagents that otherwise improve the efficiency of the assay, such as protease inhibitors, nuclease inhibitors, antimicrobial agents, etc. may be used.

The resultant mixture is incubated under conditions whereby, but for the presence of the candidate pharmacological agent, the SOUP1 polypeptide specifically binds the cellular binding target, portion or analogue with a reference binding affinity. The mixture components can be added in any order that provides for the requisite binding and incubations may be performed at any temperature, which facilitates optimal binding. Incubation periods are likewise selected for optimal binding but also minimised to facilitate rapid, high-throughput screening. Generally a plurality of assay mixtures are run in parallel with different agent concentrations to obtain a differential response to the various concentrations. Typically, one of these concentrations serves as a negative control, i.e. at zero concentration or below the limits of assay detection.

After incubation, the agent-biased binding and/or affinity between the (poly)peptide of the invention and one or more binding targets is detected by any convenient way. For cell-free binding type assays, a separation step is often used to separate bound from unbound components. The separation step may be accomplished in a variety of ways. Conveniently, at least one of the components is immobilised on a solid substrate, which may be any solid from which the unbound components may be conveniently separated. The solid substrate may be made of a wide variety of materials and in a wide variety of shapes, e.g. microtiter plate, microbead, dipstick, resin particle, etc. The substrate is chosen to maximise the signal to noise ratios, primarily to minimise background binding, for ease of washing and cost.

Separation may be effected for example, by removing a bead or a dipstick from a reservoir, emptying or diluting a reservoir such as a microtiter plate well, rinsing a bead (e.g. beads with iron cores may be readily isolated and washed using magnets), particle, chromatographic column or filter with a wash solution or solvent. Typically, the separation step will include an extended rinse or wash or a plurality of rinses and washes. For example, where the solid substrate is a microtiter plate, the wells may be washed several times with a washing solution, which typically includes those components of the incubation mixture that do not participate in specific binding such as salts, buffer, detergent, non-specific protein, etc.

Alternatively, cell-free binding type assays may be performed in homogeneous formats that do not require a separation step, e.g. scintillation proximity assay (SPA), homogenous time-resolved fluorescence assay (HTRFA). Further methods which may be employed comprise fluorescence polarisation (FP) and fluorescence resonance energy transfer (FRET).

Detection may be effected in any convenient way. For cell based assays such as one, two, and three hybrid screens, the transcript resulting from SOUP1-target binding usually encodes a directly or indirectly detectable product (e.g. galactosidase activity, luciferase activity, etc.). For cell-free binding assays, one of the components usually comprises or is coupled to a label. A wide variety of labels may be employed-essentially any label that provides for detection of bound protein. The label may provide for direct detection as radioactivity, luminescence, polarisation of light, optical or electron density, etc. or indirect detection such as an epitope tag, an enzyme, etc. The label may be appended to the protein e.g. a phosphate group comprising a radioactive isotope of phosphorous, or incorporated into the protein structure, e.g. a methionine residue comprising a radioactive isotope of sulfur.

A variety of methods may be used to detect a specific label depending on the nature of the label and other assay components. For example, the label may be detected bound to a solid substrate or a portion of the bound complex containing the label may be separated from the solid substrate, and thereafter the label detected. Labels may be directly detected through optical or electron density, radiative emission, nonradiative energy transfer, emission of polarised light, etc., or indirectly detected with antibody conjugates, etc. For example, in the case of radioactive labels, emissions may be detected directly, e.g. with particle counters or indirectly, e.g. with scintillation cocktails and counters.

A difference in the binding affinity of the (poly)peptide of the invention to the target in the absence of the agent as compared with the binding affinity in the presence of the agent indicates that the agent modulates the binding of the SOUP1 polypeptide to the SOUP1 binding target. The difference, as used herein, is statistically significant and preferably represents at least a 50%, more preferably at least a 90% difference.

Analogously, in cell-based assays, a difference in SOUP1-dependent activity in the presence and absence of an agent indicates the agent modulates SOUP1 mediated cellular function or SOUP1 expression. Such cell-based approaches may involve transient or stable expression assays. In this method, cells are transfected with one or more constructs encoding in sum, a polypeptide comprising a portion of the (poly)peptide of the invention and a reporter under the transcriptional control of an soup1 responsive promotor. The cell may advantageously also be cotransfected with a construct encoding an SOUP1 activator, e.g. a receptor capable of stimulating SOUP1 activity, etc. Alternatively, the adipose promotor itself may be linked to a suitable reporter gene, e.g. luciferase, and used in cell-based assays to screen for compounds capable of modulating, via up- or down-regulation, adipose expression.

The methods described herein are particularly suited for automated high-throughput drug screening using robotic liquid dispensing workstations. Similar robotic automation is available for high-throughput cell plating and detection of various assay read-outs.

Candidate agents shown to modulate the expression of the nucleic acid molecules of the invention or association of (poly)peptides of the invention with a binding partner provide valuable reagents to the pharmaceutical industries for animal and human trials. Target therapeutic indications are limited only in that the target soup1 cellular function (e.g. gene expression or association with a binding partner) be subject to modulation. In particular, candidate agents obtained from drug screening assays and the subject compositions, e.g. as soup1-derived nucleic acids or therapeutic polypeptides, provide therapeutic applications in diseases associated with body-weight regulation and energy homeostatis, including treatment of obesity, disorders associated with wasting, such as cancer, infectious diseases and HIV infection, or bulimia. As will be discussed herein below, for therapeutic use, the compositions and agents may be administered by any convenient way, preferably parenterally, conveniently in a physiologically acceptable carrier, e.g. phosphate buffered saline, saline, deionized water, or the like. Other additives may be included, such as stabilisers, bactericides, etc. Typically, the compositions are added to a retained physiological fluid such a blood or synovial fluid. Generally, the amount administered will be empirically determined, depending, for example, upon the therapeutic objectives, the route of administration, and the condition of the patient. Typically, the clinician will administer a molecule of the present invention until a dosage is reached that provides the required biological effect. The progress of this therapy is easily monitored by conventional assays.

A method of refining the compound or agent identified by the method of the invention comprises

(a) modeling said compound by peptidomimetics; and

(b) chemically synthesizing the modeled compound.

Peptidomimetics is well known in the art and disclosed, inter alia, in Beeley, Trends Biotech 12 (1994), 213-216, Wiley, Med. Res. Rev. 13 (1993), 327-384, Hruby, Biopolymers 43 (1997), 219-266, or references cited therein or references cited herein above.

Methods of the generation and use of peptidomimetic combinatorial libraries are described in the prior art, for example in Ostresh, Methods in Enzymology 267 (1996), 220-234 and Domer, Bioorg. Med. Chem. 4 (1996), 709-715. Methods for the chemical synthesis and/or the preparation of chemical derivatives and analogues are well known to those skilled in the art and are described in, for example, Beilstein, loc. cit. and “Organic Synthesis”, Wiley, New York, U.S.A., see supra.

It is envisaged in the present invention that the above mentioned peptidomimetics methods and/or methods for chemical synthesis, modification or for refining may also directly be employed on the compounds of the invention, e.g. on the (poly)peptides or on the fusion proteins of the invention.

The present invention relates to a method of producing a composition comprising formulating the compound of the invention, the compound or agents identified by the method(s) described herein or the compound refined by the method(s) described herein above with a pharmaceutically acceptable carrier and/or diluent.

Examples of suitable pharmaceutical carriers, excipients and/or diluents are well known in the art and include phosphate buffered saline solutions, water, emulsions, such as oil/water emulsions, various types of wetting agents, sterile solutions etc. Compositions comprising such carriers can be formulated by well known conventional methods. These pharmaceutical compositions can be administered to the subject at a suitable dose. Administration of the suitable compositions may be effected by different ways, e.g., by intravenous, intraperitoneal, subcutaneous, intramuscular, topical, intradermal, intranasal or intrabronchial administration. The dosage regimen will be determined by the attending physician and clinical factors. As is well known in the medical arts, dosages for any one patient depends upon many factors, including the patient's size, body surface area, age, the particular compound to be administered, sex, time and route of administration, general health, and other drugs being administered concurrently. Proteinaceous pharmaceutically active matter may be present in amounts between 1 ng and 10 mg per dose; however, doses below or above this exemplary range are envisioned, especially considering the aforementioned factors. If the regimen is a continuous infusion, it should also be in the range of 1 μg to 10 mg units per kilogram of body weight per minute, respectively. Progress can be monitored by periodic assessment. The compositions of the invention may be administered locally or systemically. The compositions of the invention may also be administered directly to the target site, e.g., by biolistic delivery to an internal or external target site or by catheter to a site in an artery. Preparations for parenteral administration include sterile aqueous or non-aqueous solutions, suspensions, and emulsions. Examples of non-aqueous solvents are propylene glycol, polyethylene glycol, vegetable oils such as olive oil, and injectable organic esters such as ethyl oleate. Aqueous carriers include water, alcoholic/aqueous solutions, emulsions or suspensions, including saline and buffered media. Parenteral vehicles include sodium chloride solution, Ringer's dextrose, dextrose and sodium chloride, lactated Ringer's, or fixed oils. Intravenous vehicles include fluid and nutrient replenishers, electrolyte replenishers (such as those based on Ringer's dextrose), and the like. Preservatives and other additives may also be present such as, for example, antimicrobials, anti-oxidants, chelating agents, and inert gases and the like. Furthermore, the pharmaceutical composition of the invention may comprise further agents depending on the intended use of the pharmaceutical composition.

Additionally, the present invention provides for a method of producing a composition comprising the compound(s) of the invention or the compound(s) or agent(s) identified by the method(s) of the invention comprising the steps of

(a) modifying a compound of the invention, or a compound or agent identified by the method of the invention as a lead compound to achieve

(i) modified site of action, spectrum of activity, organ specificity, and/or

(ii) improved potency, and/or

(iii) decreased toxicity (improved therapeutic index), and/or

(iv) decreased side effects, and/or

(v) modified onset of therapeutic action, duration of effect, and/or

(vi) modified pharmakinetic parameters (resorption, distribution, metabolism and excretion), and/or

(vii) modified physico-chemical parameters (solubility, hygroscopicity, color, taste, odor, stability, state), and/or

(viii) improved general specificity, organ/tissue specificity, and/or

(ix) optimized application form and route

by

(i) esterification of carboxyl groups, or

(ii) esterification of hydroxyl groups with carbon acids, or

(iii) esterification of hydroxyl groups to, e.g. phosphates, pyrophosphates or sulfates or hemi succinates, or

(iv) formation of pharmaceutically acceptable salts, or

(v) formation of pharmaceutically acceptable complexes, or

(vi) synthesis of pharmacologically active polymers, or

(vii) introduction of hydrophilic moieties, or

(viii) introduction/exchange of substituents on aromates or side chains, change of substituent pattern, or

(ix) modification by introduction of isosteric or bioisosteric moieties, or

(x) synthesis of homologous compounds, or

(xi) introduction of branched side chains, or

(xii) conversion of alkyl substituents to cyclic analogues, or

(xiii) derivatisation of hydroxyl group to ketales, acetates, or

(xiv) N-acetylation to amides, phenylcarbamates, or

(xv) synthesis of Mannich bases, imines, or

(xvi) transformation of ketones or aldehydes to Schiffs bases, oximes, acetales, ketales, enolesters, oxazolidines, thiozolidines

or combinations thereof; and

(b) formulating the product of said modification with a pharmaceutically acceptable carrier.

Pharmaceutical acceptable carriers are well known in the art, as described herein above. It is envisaged that also the compounds of the invention, i.e. the (poly)peptides or fusionproteins of the invention, the nucleic acid molecules of the invention be employed in the above described method for producing a composition. Preferably, said composition(s) is/are a pharmaceutical composition(s) as described herein.

Therefore, in a more preferred embodiment, the present invention relates to a method of producing a composition comprising the compound(s) of the invention or the compound(s) or agent(s) identified by the method(s) of the invention, wherein said composition is a pharmaceutical composition for preventing or treating obesity, adipositas, eating disorders, bulimia, wasting and/or disorders leading to increased or decreased body weight/body mass as, inter alia, described herein below.

It is particularly preferred that the present invention relates to a method for producing a composition comprising the compound(s) of the invention or compound(s) or agent(s) identified by the method(s) of the invention, wherein said composition is a pharmaceutical composition for preventing, alleviating or treating obesity, adipositas, eating disorders (like bulimia nervosa, anorexia nervosa), wasting syndromes (like cachexia), mitochondrial disorders, pancreatic dysfunctions (like diabetes), the prevention of insulin resistance, disorders related to ROS production (like response to infections, cancer, aging).

In yet another embodiment the present invention provides for a composition comprising

(a) an inhibitor of the (poly)peptide of the invention or identified by the method or refined by the method of the invention;

(b) an inhibitor of the expression of the gene identified by the method described herein or the nucleic acid molecule of the invention; and/or

(c) a compound identified by the method of the invention.

Said inhibitor of the (poly)peptide of the invention may be a compound which functions as inhibitor of the wildtype (poly)peptide of the invention, the SOUP1 protein. Said inhibitor may lead to induction of weight loss may influence regulatory cells (pancreatic beta-cells) and thereby improve beta-cell function or preventing insulin resistance, it may change ROS (reactive oxygen species) production leading to a decreased ROS concentration (causing reduced molecular damage in aging, cancerogenesis and increased ischemic tolerance). However opposite effects could occur due to tissue specific reactions and metabolic situation. Said inhibitor may also be an inhibitor specifically interacting with (a) mutated form(s) of the (poly)peptide of the invention and thereby lead to a decrease in body weight/body mass or to an maintenance of the current body weight/body mass.

It is to be understood that the term “inhibitor” of the (poly)peptide identified by the methods of the invention also relates to (an) inhibitor(s) which influence the activity and/or function of interacting (poly)peptides as identified by the method of the present invention. Said interaction may be direct or indirect. Said “inhibitor” may also interfere with and/or modify the interaction of the (poly)peptide of the invention with its binding targets/agents as defined herein. The above described applies mutatis mutandis for the term “inhibitor of the expression of the nucleic acid molecule of the invention or of the gene identified by the method(s) of the invention”. Said inhibitor may interfere with transcriptional and/or translational processes.

Similarly, the present invention relates to a composition comprising

(a) a stimulator of the (poly)peptide of the invention or of the (poly)peptide identified or refined by the method(s) described herein above;

(b) a stimulator of the expression of the nucleic acid molecule of the invention or of the gene identified by the method(s) of the invention;

(c) a compound identified by the method(s) of the invention; and/or

(d) the vector of the invention.

The term “stimulation of the (poly)peptide” of the invention relates to a compound which functions as a stimulator (activator) of the (poly)peptides of the invention. Said stimulator/activator may lead to a induction of weight gain and may be useful for the treatment of wasting. It may also change the ROS production which may lead to increased efficacy in (a) immune response(s). Yet, opposite effects are also envisaged, due to tissue specific reactions and metabolic situations. The here described “stimulators” may, inter alia, lead to an increased interaction of the (poly)peptide of the invention with its binding target. The term also relates to a stimulator/activator of the mutated form(s) of the (poly)peptides of the present invention. Said stimulator(s) of the mutated form(s) may lead to an increase in body weight/body mass or to an maintenance of the current body weight/body mass.

“Inhibitors” as well as “stimulators of the (poly)peptide of the invention” may be deduced and/or evaluated by methods known in the art and disclosed herein.

The term “stimulator of a (poly)peptide identified or refined by the method(s) of the present invention” relates also to a stimulator which influences the activity/function of (interacting) (poly)peptides as identified by the method of the present invention they may interact with said (poly)peptides in either direct or indirect fashion. As already mentioned for the term “inhibitor” as defined herein above, the above said applies, mutatis mutantis, for the term “stimulator of the expression of the nucleic acid molecule of the invention or of the gene identified by the method(s) of the invention”.

The above mentioned “inhibitors” and “stimulators” not only relate to (poly)peptides, but may also comprise small molecules which bind to, interfere with and/or interact with the (poly)peptides and/or nucleic acid molecules of the invention or with (poly)peptides and/or genes identified by the method(s) of the invention. Examples of such small molecules comprise, but are not limited to small peptides, anorganic and/or organic substances or peptide-like molecules, like peptide-analogs comprising D-amino acids. Said “inhibitors” and “stimulators” may further comprise antibodies, derivatives and/or fragments thereof, aptamers or specific (oligo)nucleotides. The “inhibitors” and “stimulators” may also be part of the pharmaceutical and/or diagnostic compositions as disclosed herein.

As pointed out herein above, said “inhibitorst” or “stimulators” may also comprise small organic compounds as defined herein above.

In addition, the present invention relates to a composition comprising a nucleic acid molecule of the invention, a (poly)peptide of the invention, a fusionprotein of the invention, an antibody or (a) fragment(s) or derivative(s) thereof or an aptamer of the invention or an anti-sense oligonucleotide of the invention. Furthermore, said composition may comprise (poly)peptides, nucleic acid molecules, genes and/or compounds or agents as identified by the methods of the present invention.

In a preferred embodiment of the invention, said composition is a pharmaceutical, e.g. therapeutic composition. Pharmaceutical compositions comprising, optionally, pharmaceutically acceptable carriers have been described herein above. The pharmaceutical compositions of the present invention are particularly useful for the treatment and/or the prevention of complex disorders of appetite regulation and/or energy metabolism.

It is particularly preferred that said pharmaceutical composition is employed in treating and/or preventing obesity, adipositas, eating disorders, bulimia, disorders of body weight/body mass. It is, however, also envisaged that said pharmaceutical compositions be used in disorders like, inter alia, wasting (eachexia), weight loss due to cancer or infectious diseases or weight loss in immuno-compromised patients, like, HIV-patients.

It is furthermore envisaged that the pharmaceutical composition of the invention may be used in combination with other agents employed in the treatment of body weight/mass disorders. Said agents may comprise, but are not limited to, agents reducing/enhancing food intake, agents blocking/activating nutrient absorption, agents increasing/decreasing thermogenesis, agents modulating fat and/or protein metabolism or storage, agents modulating the central controller regulating body weight Said agents may, inter alia, comprise, agents like sibutramine, orlistat, ephedrine or caffeine, diethylpropione, phentermine, fluoxetine, sertraline, or phenylpropanolamine.

Furthermore, the present invention relates to a composition comprising a nucleic acid molecule of the invention, a (poly)peptide of the invention, a fusionprotein of the invention, an antibody, a derivative or fragment thereof an aptamer of the invention at least a primer or a set of primers as defined herein or an anti-sense oligonucleotide of the invention. Particularly preferred primers are primers as employed in the appended examples and/or depicted in SEQ ID NOs. 55 to 60.

It is, e.g., envisaged that primers deduced from the nucleic acid molecules of the invention are employed for diagnostic or scientific purposes. Said primers may, inter alia, be employed to find and/or verify mutations of SOUP1 genes in individuals. Preferably, said individuals are humans. Furthermore, primers as deduced from the nucleic acid sequences disclosed herein, in particular from sequences as shown in SEQ ID NOs: 9, 11, 13 and 51 may be employed/used to detect and/or isolate homologous sequences in further species.

In a particular preferred embodiment said composition is a diagnostic composition. Said diagnostic composition may comprise the components as defined herein above wherein said components are bound to/attached to and/or linked to a solid support as defined herein above. It is furthermore envisaged, that said diagnostic composition comprises a compound(s) of this invention on (micro-)chips. Therefore, said diagnostic composition may, inter alia, comprise the nucleic acid molecules of the invention on so-called “gene chips” or the (poly)peptides of the invention on so-called “protein-chips”. Diagnostic gene chips may comprise a collection of the nucleic acid molecules of the invention that, e.g., specifically detect mutations in the SOUP1-gene of animals, in particular of humans. Said diagnostic compositions and in particular the diagnostic gene chip as described herein above may be particularly useful for screening patients for (genetic) defects underlying, e.g. obesity, adipositase, disorders of body weight/body mass, or eating disorders.

It is preferred that said compounds of the present invention to be employed in a diagnostic composition are detectably labeled. A variety of techniques are available for labeling biomolecules, are well known to the person skilled in the art and are considered to be within the scope of the present invention. Such techniques are, e.g., described in Tijssen, “Practice and theory of enzyme immuno assays”, Burden, R H and von Knippenburg (Eds), Volume 15 (1985), “Basic methods in molecular biology”; Davis L G, Dibmer M D; Battey Elsevier (1990), Mayer et al., (Eds) “Immunochemical methods in cell and molecular biology” Academic Press, London (1987), or in the series “Methods in Enzymology”, Academic Press, Inc.

There are many different labels and methods of labeling known to those of ordinary skill in the art. Examples of the types of labels which can be used in the present invention include enzymes, radioisotopes, colloidal metals, fluorescent compounds, chemiluminescent compounds, and bioluminescent compounds.

Furthermore, the present invention relates to the use of

(a) an inhibitor of the (poly)peptide identified or refined by the method of the invention;

(b) an inhibitor of the expression of the gene identified by the method of the invention; and/or

(c) a compound identified by the method of the invention;

for the preparation of a pharmaceutical composition for the treatment of obesity, adipositas, eating disorders, wasting syndromes (e.g. cachexia), mitochondrial disorders, pancreatic dysfunctions, disorders related to ROS production.

Similarly, the present invention also provides for the use of

(a) a stimulator of the (poly)peptide identified or refined by the method of the invention;

(b) a stimulator of the expression of the gene identified by the method of the invention; and/or

(c) a compound identified by the method of the invention;

for the preparation of a pharmaceutical composition for the treatment of obesity, adipositas, eating disorders, wasting syndromes (cachexia), mitochondrial disorders, pancreatic dysfunctions, disorders related to ROS production.

Furthermore, the present invention relates to the use of an agent as identified by the method of the invention for the preparation of a pharmaceutical composition for the treatment, alleviation and/or prevention of obesity, adipositas, eating disorders, wasting syndromes (cachexia, also in cancer, HIV-infections), mitochondrial disorders as described herein, pancreatic dysfunctions (like diabetes), disorders related to ROS production (like cancer, aging, infections).

In addition, the present invention relates to the use of a nucleic acid molecule as depicted in SEQ ID NOs: 3 or 4 (dUCP) or of (a) fragment(s) thereof for the preparation of a non-human animal which over- or underexpresses the gene product as encoded by SEQ ID NOs: 3 or 4 or (a) fragment(s) thereof. Said non-human animal is preferably a fruit fly. The use as described herein above is illustrated in the appended examples.

In a particular preferred embodiment, the present invention relates to the use of a fruit fly as defined in herein above for the detection of polypeptides capable of contributing to membrane stability and/or function in organelles, capable of modifying mitochondrial proteins, and/or capable of influencing cellular metabolism.

Furthermore, the invention provides for a kit comprising at least one of a nucleic acid molecule, a vector, a host, a polypeptide, a fusion protein, an antibody or a fragment or derivative thereof or an antiserum, an aptamer or another receptor and an anti-sense oligonucleotide of the invention.

Advantageously, the kit of the present invention further comprises, optionally (a) reaction buffer(s), storage solutions and/or remaining reagents or materials required for the conduct of scientific or diagnostic assays or the like. Furthermore, parts of the kit of the invention can be packaged individually in vials or bottles or in combination in containers or multicontainer units.

The kit of the present invention may be advantageously used, inter alia, for carrying out the method of producing a (poly)peptide of the invention and could be employed in a variety of applications referred herein, e.g., as diagnostic kits, as research tools or vaccination tools. Additionally, the kit of the invention may contain means for detection suitable for scientific medical and/or diagnostic purposes. The manufacture of the kits follows preferably standard procedures which are known to the person skilled in the art.

The examples illustrate the invention.

### EXAMPLE 1

### Cloning of a Drosophila melanogaster Gene with Homology to Human Uncoupling Proteins (UCPs)

A BLAST homology search was performed in a public database (NCBI/NIH) looking for Drosophila genes with sequence homology to the human UCP2 and UCP3 genes. The search yielded sequence fragments of a family of Drosophila genes with UCP homology. They are clearly different to the next related mitochondrial proteins (oxoglutarate carrier).

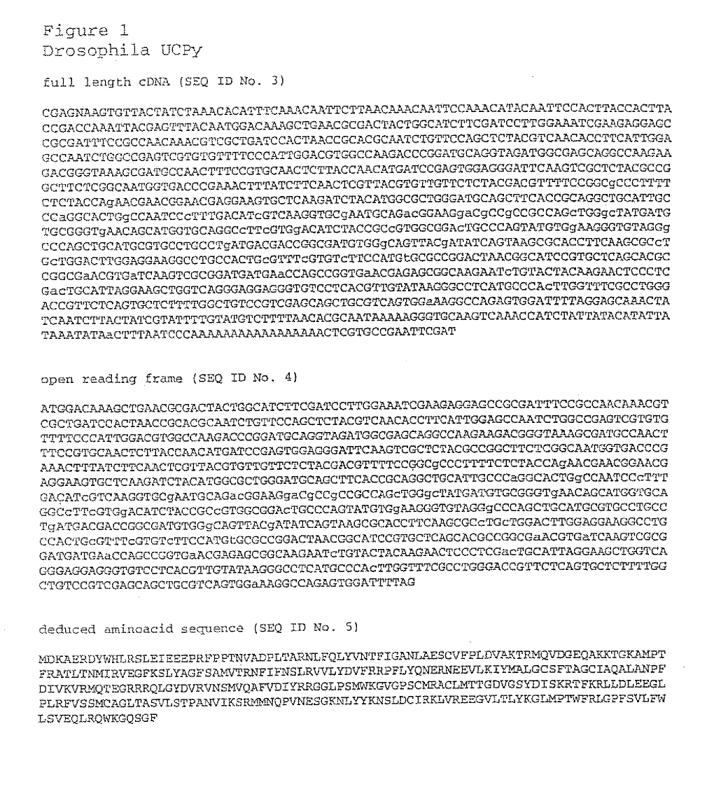

Using the sequence fragment of one of this genes (here called dUCPy) a PCR primer pair was generated (Upper 5′-CTAAACAAACAATTCCAAACATAG (SEQ ID NO: 1), Lower 5′-AAAAGACATAGAAAATACGATAGT (SEQ ID NO: 2)) and a PCR reaction performed on Drosophila cDNA using standard PCR conditions. The amplification product was radioactively labelled and used to screen a cDNA library made from adult Drosophila flies (Stratagene). A full-length cDNA clone was isolated, sequenced (FIG. 1) and used for further experiments.

### EXAMPLE 2

### Cloning of the dUCPy cDNA into an Drosophila Expression Vector

In order to test the effects of dUCPy expression in Drosophila cells the dUCPy cDNA was cloned into the expression vector pUAST (Ref.: Brand A & Perrimon N, Development 1993, 118:401-415) using the restriction sites NotI and Kpn1. The resulting expression construct was injected into the germline of Drosophila embryos and Drosophila strains with a stable integration of the construct were generated. Since the expression vector pUAST is activated by the yeast transcription factor Gal4 which is normally absent from Drosophila cells dUCPy is not yet expressed in these transgenic animals. If pUAST-dUCPy flies are crossed with a second Drosophila strain that expresses Gal4 in a tissue specific manner the offspring flies of this mating will express dUCPy in the Gal4 expressing tissue.

The cross of pUAST-dUCPy flies with a strain that expresses Gal4 in all cells of the body (under control of the actin promoter) showed no viable offspring. This means that dUCPy overexpression in all body cells is lethal. This finding is consistent with the assumption that dUCPy overexpression could lead to a collapse of the cellular energy production.

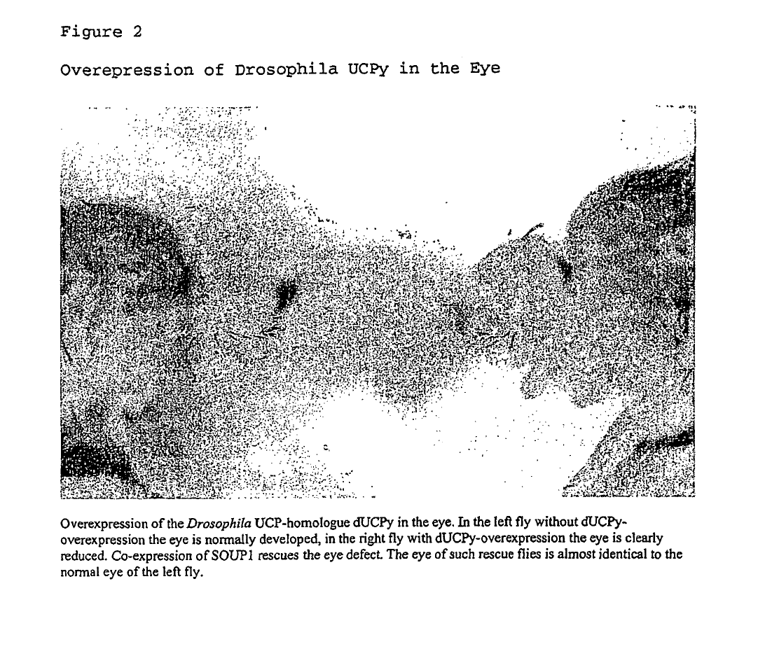

Expression of dUCPy in a non-vital organ like the eye (Gal4 under control of the eye-specific promoter of the “eyeless” gene) results in flies with visibly damaged eyes (FIG. 2). This easily visible eye phenotype is the basis of a genetic screen for gene products that can modify UCP activity.

### EXAMPLE 3

### dUCPy Modifier Screen

Parts of the genomes of the strain with Gal4 expression in the eye and the strain carrying the pUAST-dUCPy construct were combined on one chromosome using genomic recombination. The resulting fly strain has eyes that are permanently damaged by dUCPy expression. Flies of this strain were crossed with flies of a large collection of mutagenized fly strains. In this mutant collection a special expression system (EP-element, Ref.: R¢rth P, Proc Natl Acad Sci USA 1996, 93(22):12418-22) is integrated randomly in different genomic loci. The yeast transcription factor Gal4 can bind to the EP-element and activate the transcription of endogenous genes close the integration site of the EP-element. The activation of the genes therefore occurs in the same cells (eye) that overexpress dUCPy. Since the mutant collection contains several thousand strains with different integration sites of the EP-element it is possible to test a large number of genes whether their expression interacts with dUCPy activity. In case a gene acts as an enhancer of UCP activity the eye defect will be worsened; a suppressor will ameliorate the defect.

Using this screen a novel gene with suppressing activity was discovered that is here called Suppressor Of Uncoupling Protein (SOUP1). Expression of SOUP1 together with dUCPy in the Drosophila eye leads to a rescue of the dUCPy induced defect (FIG. 2).

### EXAMPLE 4

### Cloning of SOUP1 from Drosophila (dSOUP1)

Genomic DNA neighbouring to the eye-defect rescuing EP-element was cloned and sequenced. This sequence was used for a BLAST search in a public Drosophila gene database. A short sequence fragment (EST) from cDNA clone

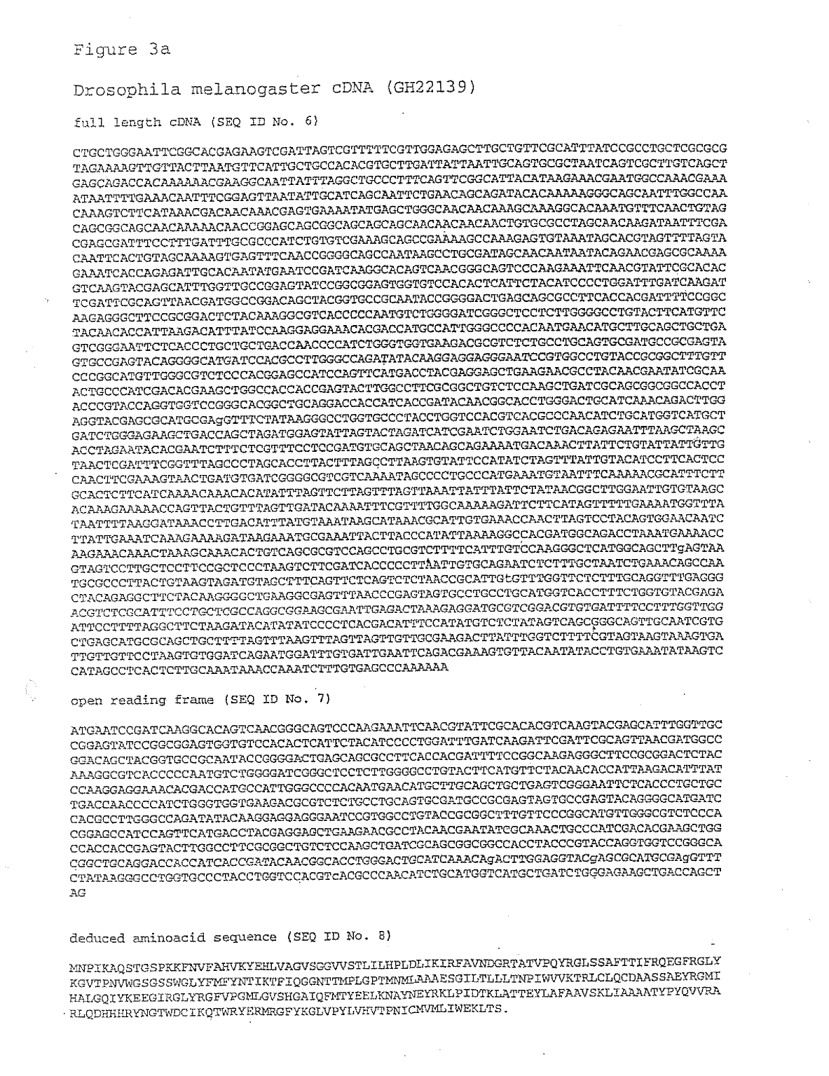

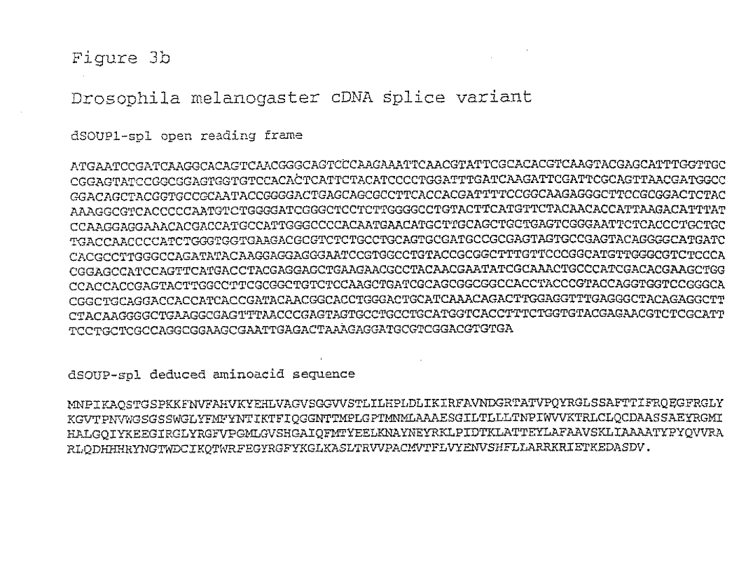

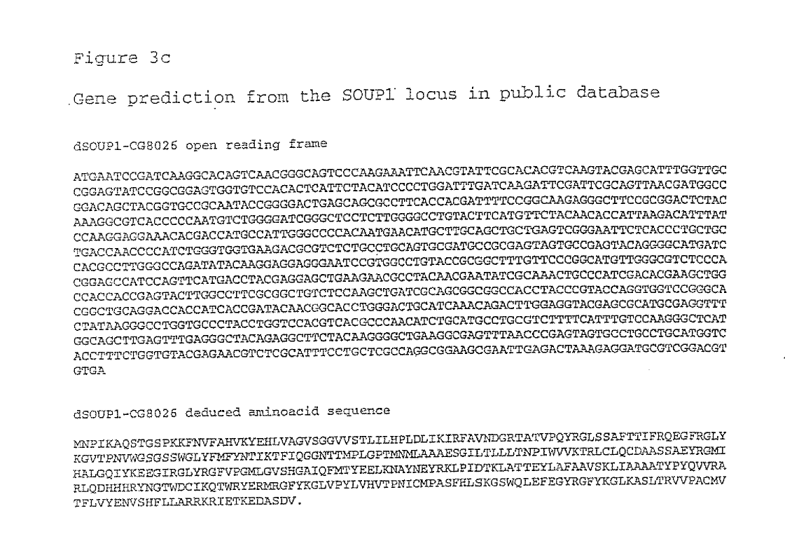

GH22139 (Drosophila Genome Project) with identical sequence was discovered in the database search. This publicly available cDNA clone was ordered and then fully sequenced (FIG. 3a).

### EXAMPLE 5

### Overexpression of GH22139 in the Drosophila eye

To ensure that GH22139 encodes for the genetically identified dUCPy suppressor SOUP1 the GC22139 cDNA was cloned into the expression vector pUAST (using restriction sites BglII and XhoI). By means of germ line injection transgenic animals were generated. pUAST-GH22139 transgenic flies were crossed with the flies expressing dUCPy in the eye. In the offspring of this cross (flies expressing dUCPy and GH22139) the eye defect was rescued. This proved that the CG22139 cDNA encodes for SOUP1.

### EXAMPLE 6

### Sequence analysis of dSOUP1

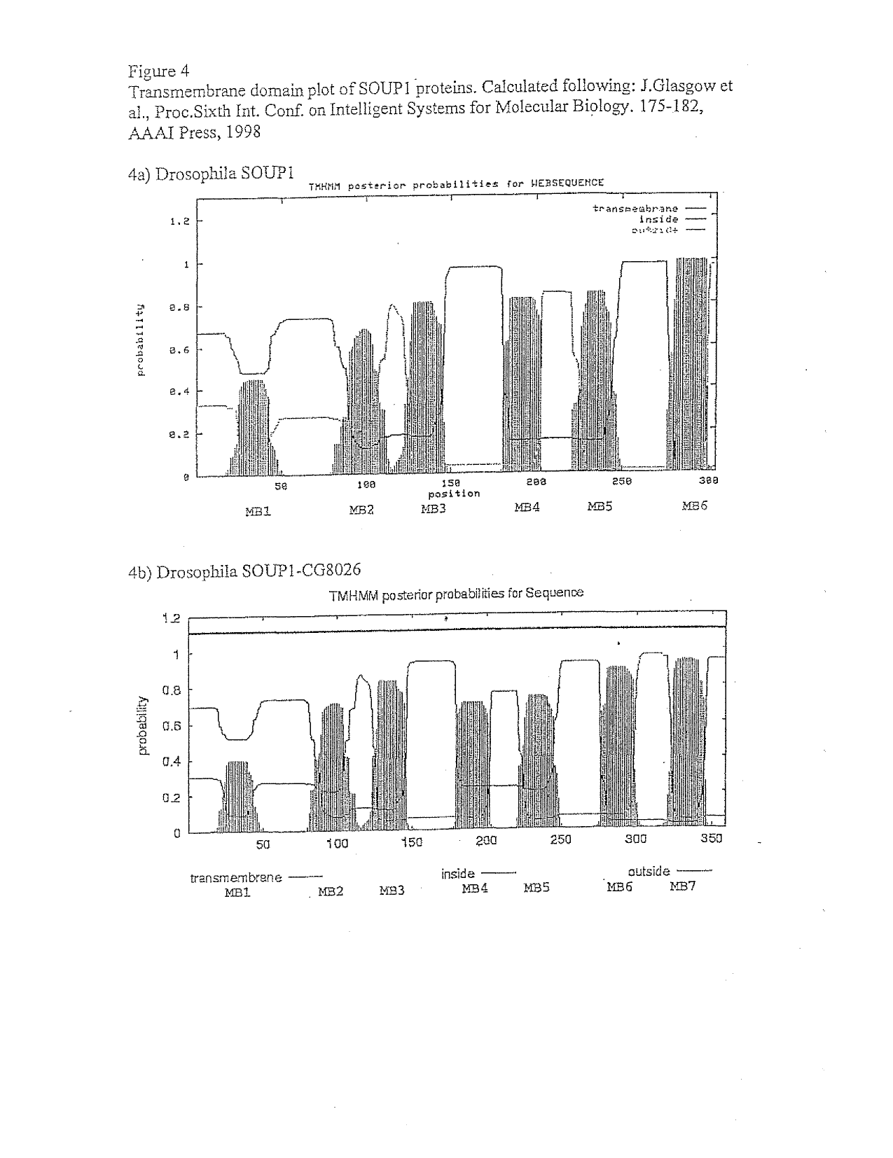

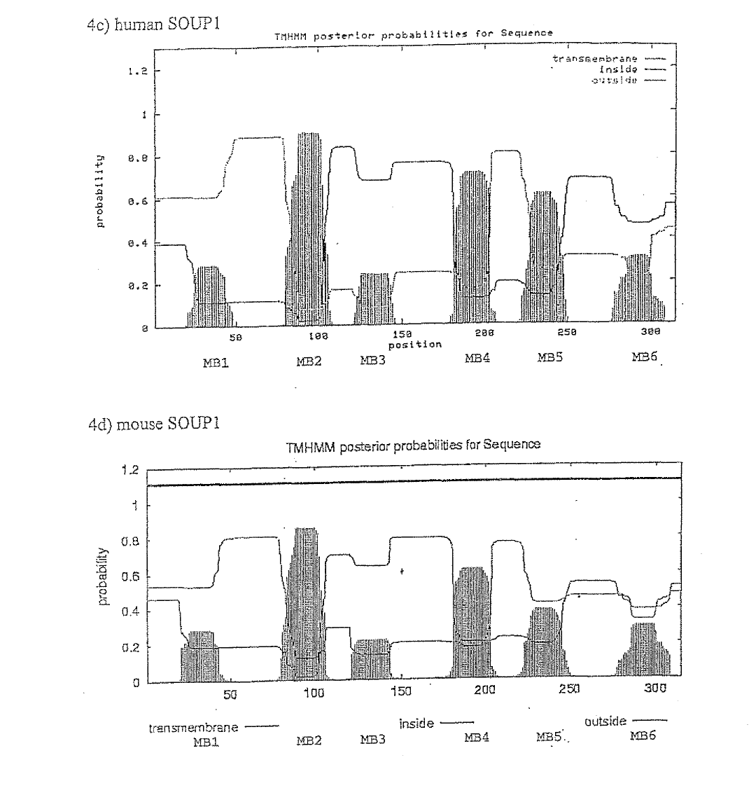

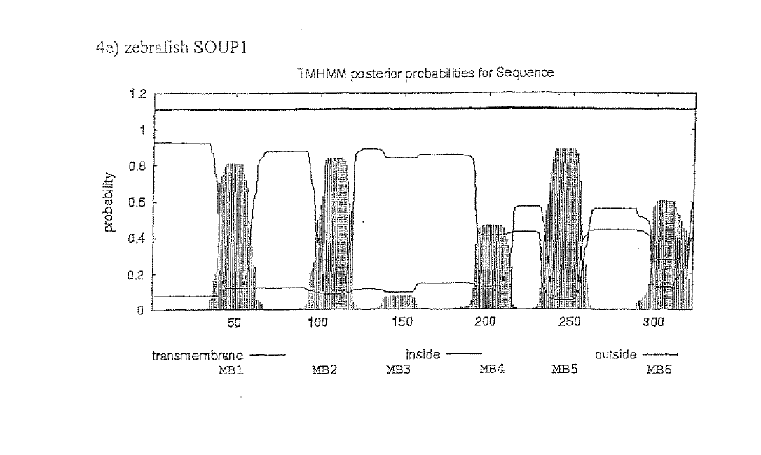

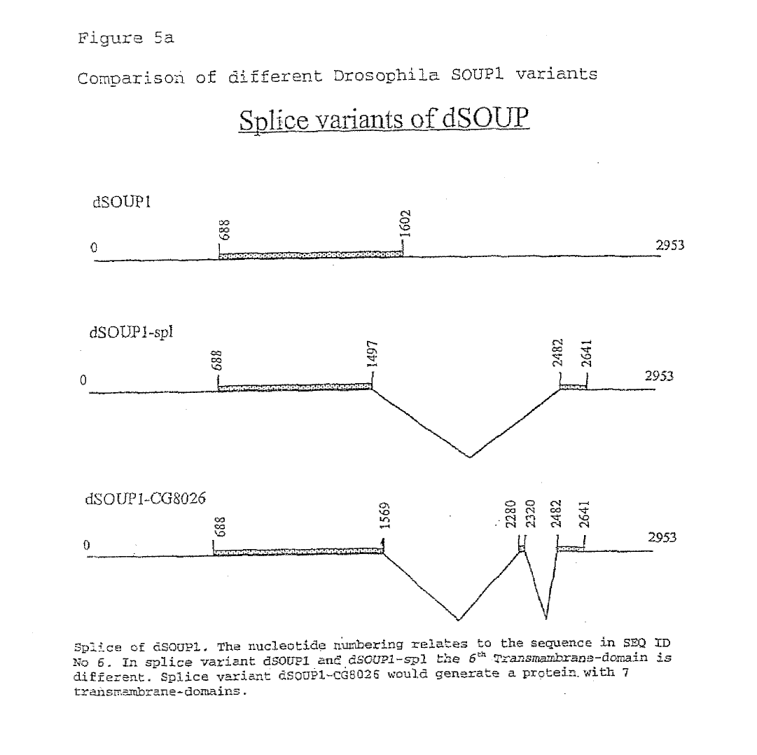

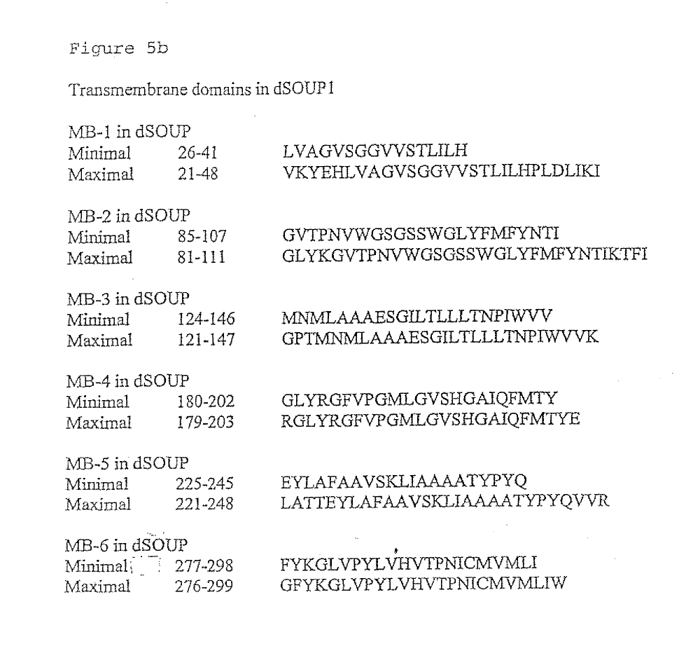

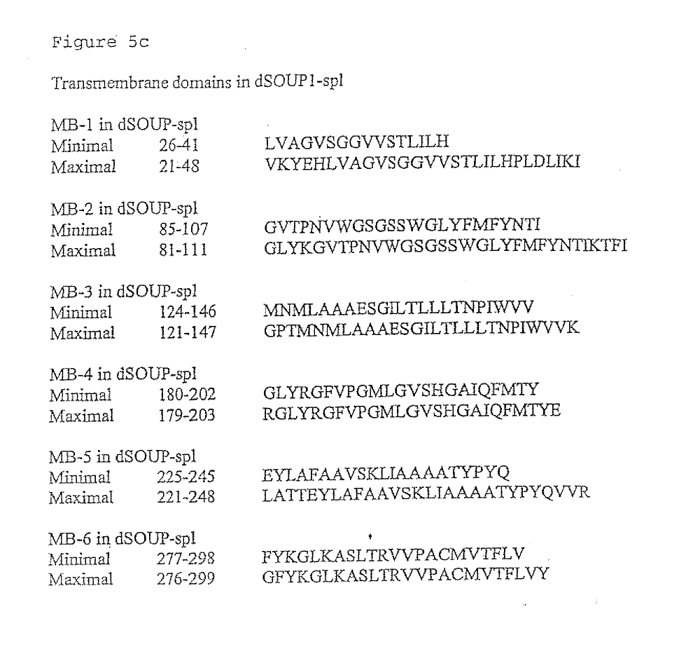

The analysis of the deduced aminoacid sequence of the SOUP1 cDNA with bioinformatic tools (Ref.: J. Glasgow et al., Proc. Sixth Int. Conf. on Intelligent Systems for Molecular Biology. 175-182, AAAI Press, 1998) showed that the SOUP1 protein has the characteristic pattern of a mitochondrial carrier protein with 6 transmembrane domains (FIG. 4a).

### EXAMPLE 7

### Possible Splice Variants of dSOUP1 and Comparison with a Predicted Gene in Public Databases

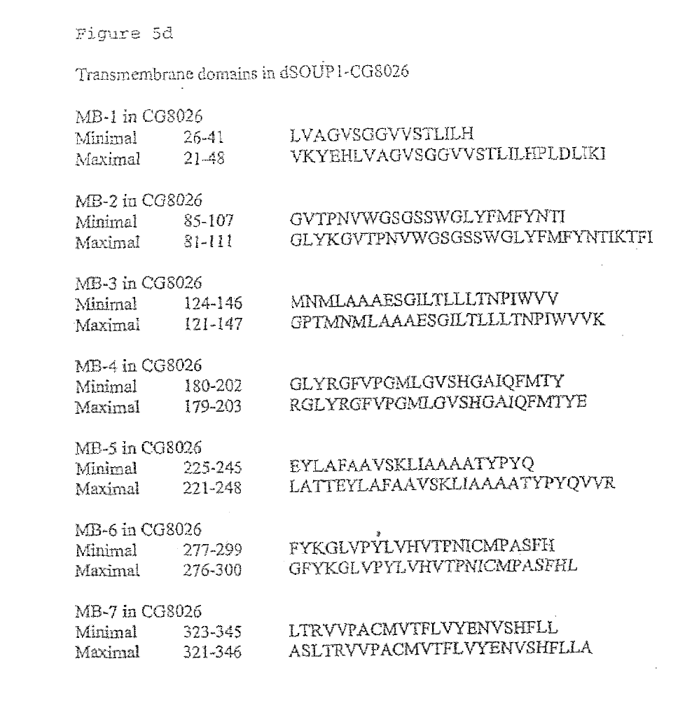

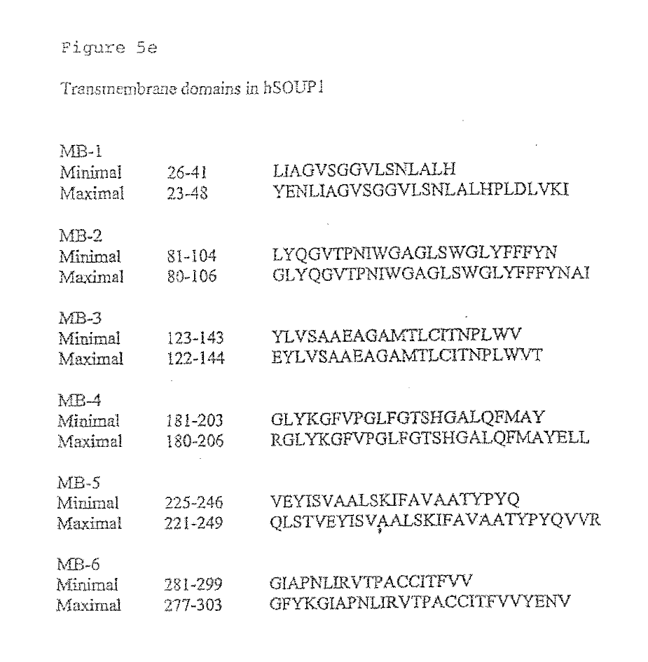

Sequence alignments, transmembrane-domain predictions and splice-predictions suggest that the Drosophila SOUP1 gene exists in different splice variants (FIG. 3b, c and FIG. 5a). The cDNA clone CG22139 can form two splice variants (dSOUP1 and dSOUP1-sp1) with a different 6^{th} TM-domain. Searching the fully sequenced Drosophila genome in public databases revealed a gene prediction (dSOUP1-CG8026, Ref. Adams, M. D. et al., 2000 Science 287:2185) that suggests a splice variant with 7 TM-domains. The homologies between the variant are:

amino acid homology in % (identity/similarity)
dSOUP1sp1-dSOUP1-CC8026 (82/82)
dSOUP1sp1-dSOUP1-CC8026 (89/89)

dSOUP1-dSOUP1sp1 (93/94)

The amino acids of the predicted TM-domains of the different splice variant an species are depicted in FIG. 5b to t).

### EXAMPLE 8

### Cloning of SOUP1 homologues from human (hSOUP1), mouse (mSOUP1), and zebrafish (zSOUP1)

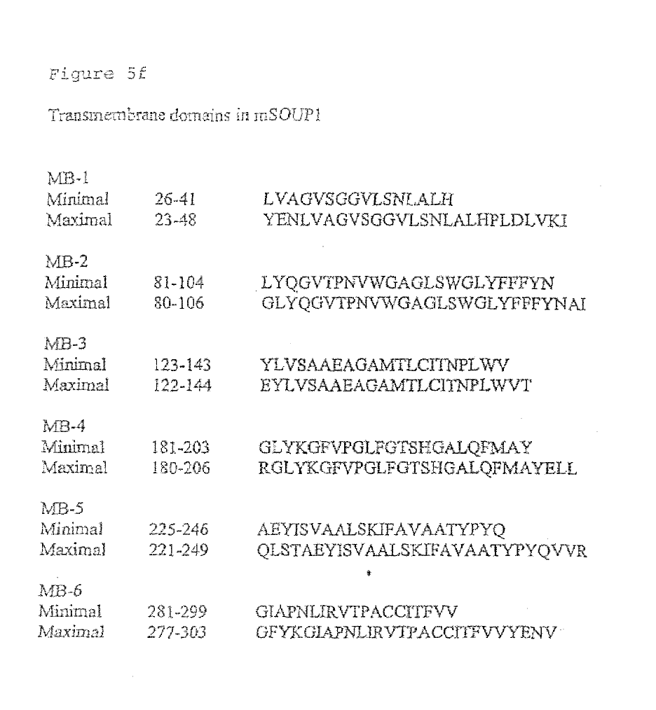

Using the complete cDNA sequence of Drosophila SOUP1 a BLAST search in a public database was performed. Several short, uncomplete sequence fragments from different species (zebra fish, chick, mouse, macaque, human) were found. Since none of the sequences in the database were complete the human sequence fragment was used to synthesize PCR-primers. The PCR amplification product was radioactively labeled and used as a probe to screen a human adipocyte cDNA library and a human hippocampus cDNA library. From both libraries cDNA clones could be isolated. The sequence of the human SOUP1 is shown in FIG. 6. It shows the characteristic 6 transmembrane domain pattern as well (FIG. 4c). The following PCR primers can be used to amplify the ORF of the human SOUP1 gene: Upper 5′-ATG GAC TAC GGG GAC TTT ATC AA (SEQ ID NO: 55), Lower 5′-ACC GAC GCC TTC TTT CCA ACT (SEQ ID NO: 56).

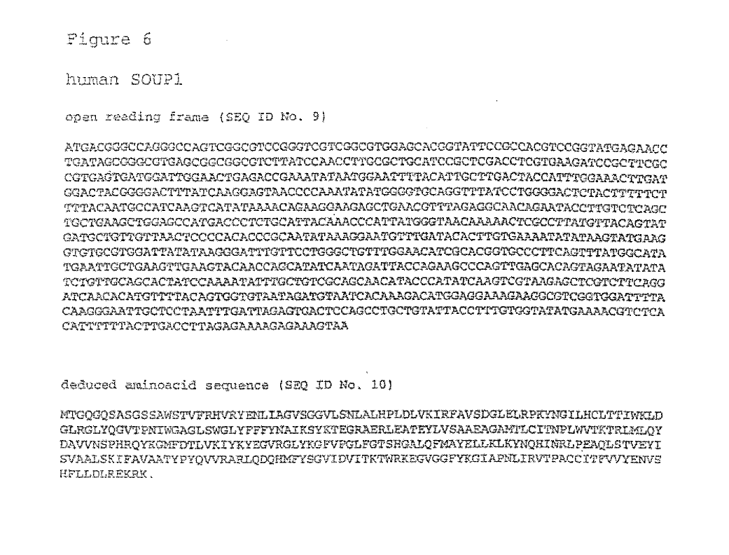

The identification of the mouse Soup1 gene was done in an equivalent approach. The sequence of mSOUP1 is shown in FIG. 7. It shows the characteristic 6 transmembrane domain pattern as well (FIG. 4d). The following PCR primers can be used to amplify the ORF of the mouse SOUP1 gene: Upper 5′-GGC GGC CAC TAC ATC ACG (SEQ ID NO: 57), Lower 5′-TGCCTCAAGAACATAGACTG (SEQ ID NO: 58).

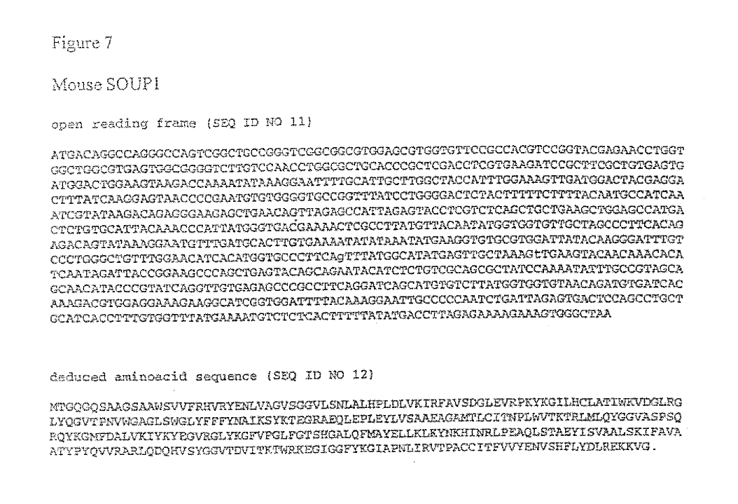

During the isolation of mouse SOUP1 cDNA clones the clone 61 was isolated that has an alternative sequence. The sequence of the open reading frame is shown in FIG. 8. The deduced amino-acid sequence differs in three positions from the other mouse SOUP1 clone.

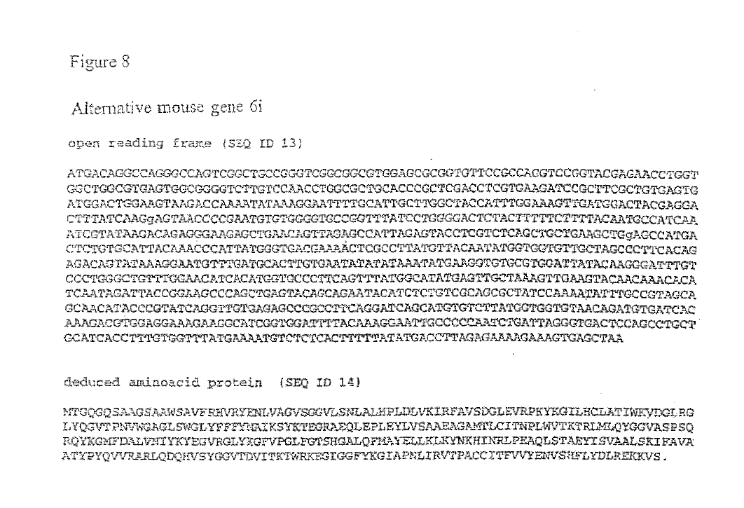

The identification of the zebrafish Soup1 gene was done in an equivalent approach. The sequence of zSOUP1 is shown in FIG. 9. It shows the characteristic 6 transmembrane domain pattern as well (FIG. 4e). The following PCR primers can be used to amplify the ORF of the zebrafisch SOUP1 gene: Upper 5′-ATGACCGCTACCATTTCCAGGCAGAGGCAC (SEQ ID NO: 59), Lower 5′-TTAGTGATACTCGCCCAAAAGCAAGCGTGA (SEQ ID NO: 60).

### EXAMPLE 9

### Generation of a Flag-Tagged mSOUP Transgene

A carboxy-terminally flag-tagged mSOUP transgene (herein referred to as mSOUPFlag) was generated by performing RT-PCR on cDNA derived from mouse pancreas RNA using mSOUP gene specific primers (mSOUP forward primer: 5′ GGCGGCCACTACATCACG3′ (SEQ ID NO:63), flag-tagged mSOUP reverse primer: 5′CTACTTGTCATCATCGTCCTTGTAGTCGCTCACTTTCTTTTCTCT 3′ (SEQ ID NO:64)).

### EXAMPLE 10

### Generation of Beta-Actin-mSOUPFlag Transgenic Mice

The mSOUPFlag (see Example 9) was expressed in mice under the control of the ubiquitous human beta-actin promoter using techniques known to those skilled in the art (for example, see, Gunnig et al. (1987). Proc. Natl. Acad. Sci. USA 84, 4831-4835). A transgenic construct DNA was injected into C57/BL6 mouse embryos (Harlan Winkelmann, Borchen, Germany) using standard techniques (see, for example, Brinster et al. (1985). Proc. Natl. Acad. Sci. USA 82, 4438-4442). Using this technique, eight independent mouse founderlines were generated.

### EXAMPLE 11

### mSOUP Expression Analysis via TaqMan Analysis

The expression of the mSOUPFlag transgene was monitored by TaqMan analysis. For this analysis, 1-100 ng, preferably 50 ng cDNA derived from different mouse tissues (for example, colon, heart, liver, lung, stomach, intestine, white adipose tissue (WAT), thymus, spleen, muscle, kidney) and a mSOUP specific primer/probe pair were used (mSOUP forward primer (SEQ ID NO: 65): 5′-CTCTGTCGCAGCGCTATCC-3′, mSOUP reverse primer (SEQ ID NO: 66): 5′-GAAGGCGGGCTCTCACAAC-3′, mSOUP probe (SEQ ID NO: 67): 5′-AATATGCCGTAG CAGCAACATACCCGT-3′).

TaqMan analysis was performed using standard techniques known to those skilled in the art.

In the eight independent mouse founder lines analysed, ectopic mSOUP expression using wild-type mice as a reference was detected in all tissues apart from brown adipose tissue (BAT). The two founder lines showing highest transgene expression levels were used for further analysis.

### EXAMPLE 12

### mUCP2 Expression Analysis Via Northern Blot Analysis

To study the effect of ubiquitous, ectopic mSOUPFlag expression in a transgenic mouse model in vivo, mUCP2 expression in actin-mSOUPFlag transgenic mice (and in control littermate) was monitored by Northern blot analysis using 20 g total RNA and a 1.0 kb mUCP2 cDNA encompassing the complete UCP2 open reading frame as a probe. To ensure equal loading, the Northern blot was rehybridized with a 1.4 kb human-actin cDNA as a probe. Northern blot analysis was performed using standard techniques (see, for example, Sambrook et al. (1989) Molecular Cloning, a laboratory manual. Second Edition, Cold Spring Harbor Laboratory Press).

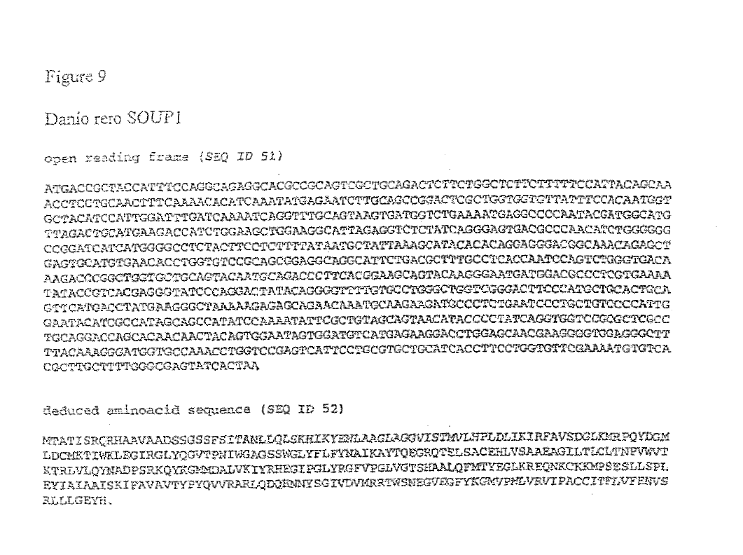

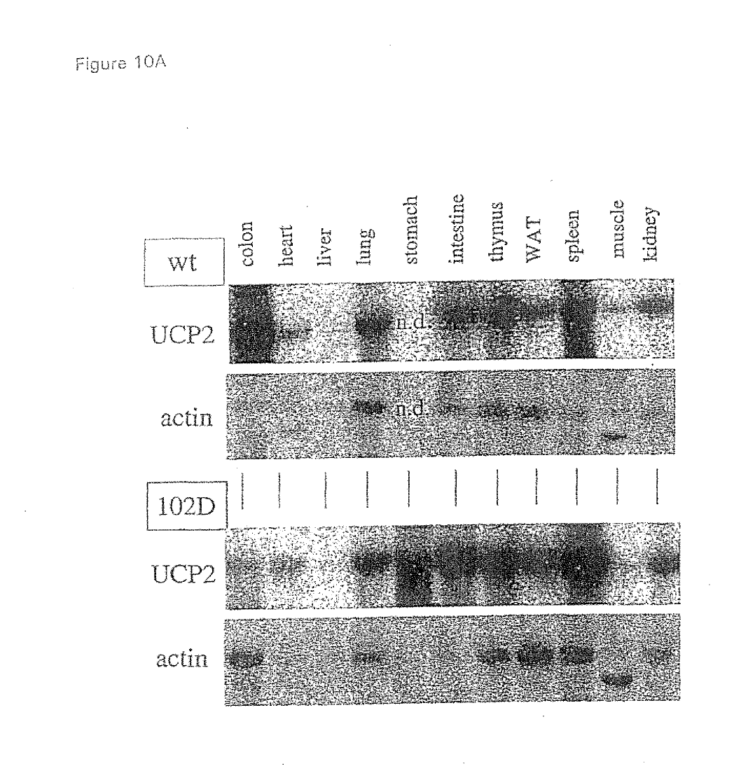

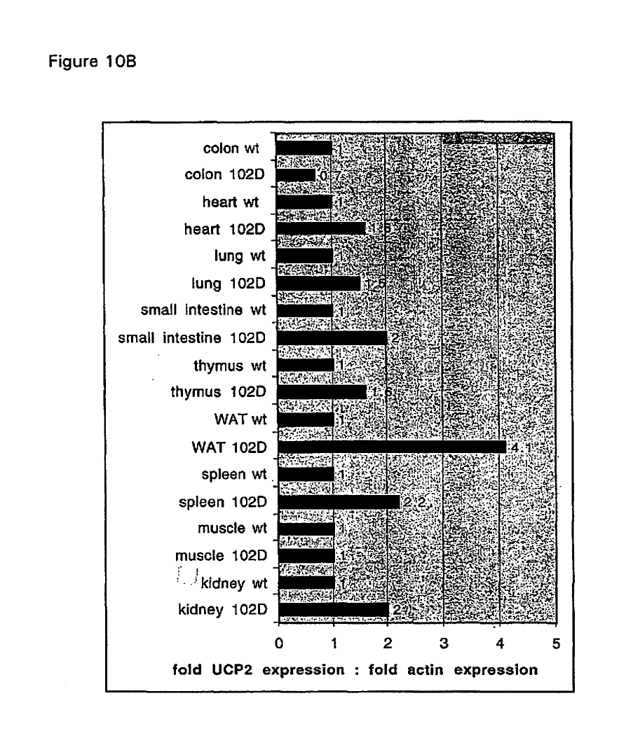

Expression of mouse UCP2 was detected in all tissues tested (see FIG. 10). Comparison of mUCP2 expression levels in transgenic mice relative to control littermates revealed increased mUCP2 expression in several different tissues analysed.

Quantification analysis using an Instant Imager and corresponding analysis software showed 1.5-4.1 fold increased mUCP2 expression in lung, small intestine, thymus, WAT, spleen and kidney of actin-mSOUPFlag transgenic mice relative to the wild-type control littermates (see FIG. 10B)

### EXAMPLE 13

### Fluorescence Microscopy Analysis of Mouse Soup Localisation in NIH3T3 Cells

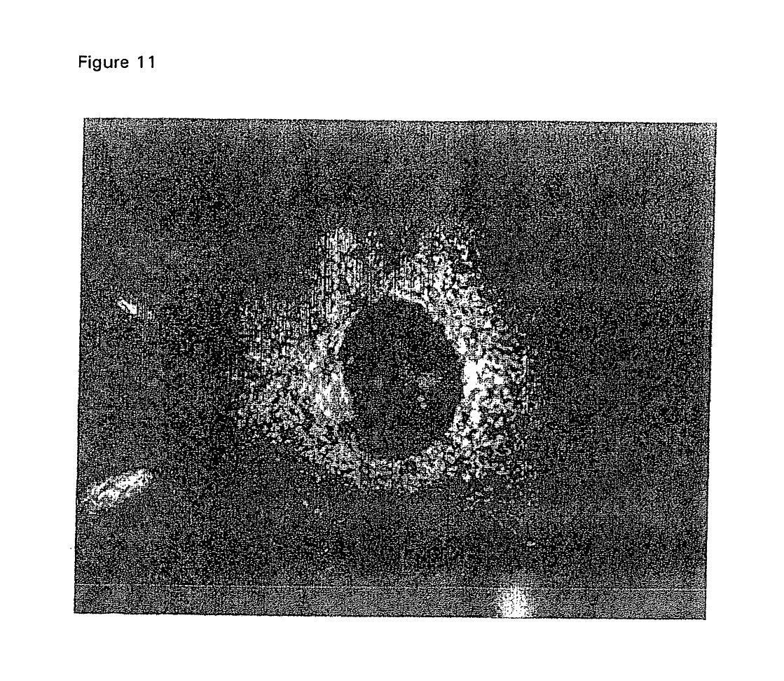

NIH 3T3 cells were transiently transfected with an expression vector for mouse Soup, fixed und immunostained with an antisera against mouse Soup. The immunofluorescence was examined at 630× magnification and appropriate filters for the Cy3-labelled secondary antibody. It was shown that Soup is cleared localized in the mitochondria of NIH 3T3 cells (SEE FIG. 11).

NIH3T3 cells were seeded into 24 wells plates containing Poly-D-Lysine coated coverslips (BD Biosciences, Erembodegem, Belgium) at 25.000 cells per well. The day after seeding, cells were transiently transfected with the Soup expression construct under the control of a CMV-Promoter with Lipofectamin Plus (InvitroGen, Karlsruhe, Germany) according to the manufacturer's instructions. 48 hours after transfection, cells were fixed in 4% para-formaldehyde and immunostained according to Dorner et al (1998) J Biol. Chem. 273: 20267-75). In brief cells were permeabilized with 0.75% Triton X-100 for 10 minutes in PBS, endogenous autofluorescence blocked by treatment of the cells with 0.1 NaBH₄ in PBS for 10 minutes. After a short PBS wash, cell were incubated in blocking buffer (PBS, 0.5% BSA, 5% goat serum, 0.045% fish gelatine) for 1 hour. Primary antisera (1:25, overnight) and secondary antibody (anti-rabbit-Cy3, 1:200, 1 hour) were applied in blocking buffer followed by washes in blocking buffer. The cover slips were mounted on glass slides and immunostained cells were examined in an fluorescence microscope with the appropriate filter set for Cy3.

### EXAMPLE 14

### Anti mSOUP Antibodies

Anti mouse SOUP antibodies were raised in rabbits by immunizing with the synthetic peptide SEQ ID NO:68: CYENVSHFLYDLREKKVS, corresponding to the C-terminus of mouse SOUP protein (C-a.a. 300-316 from SEQ ID NO:14). The antibodies were affinity purified using a SULFO-Link column (Order No. Pierce 44895) carrying the synthetic peptide SEQ ID NO:68: (CYENVSHFLYDLREKKVS), according to the manufacturers' protocol.

### EXAMPLE 15

### Effect of SOUP1 mRNA inhibition in adipocytes

An RNAi sense/antisense construct was introduced into 3T3-L1 adipocytes:

### Construct:

mSOUP III: ko.sense strand

(SEQ ID NO: 69)

5′ GATCCCCTAGC AGCAACATAC CCGTATTCAA GAGATACGGG
TATGTTGCTG CTATTTTTGG AAA 3′

mSOUP-III: ko.antisense strand

(SEQ ID NO: 70)

5′ AGCTTTTCCA AAAATAGCAG CAACATACCC GTATCTCTTG
AATACGGGTA TGTTGCTGCT A GGG 3′

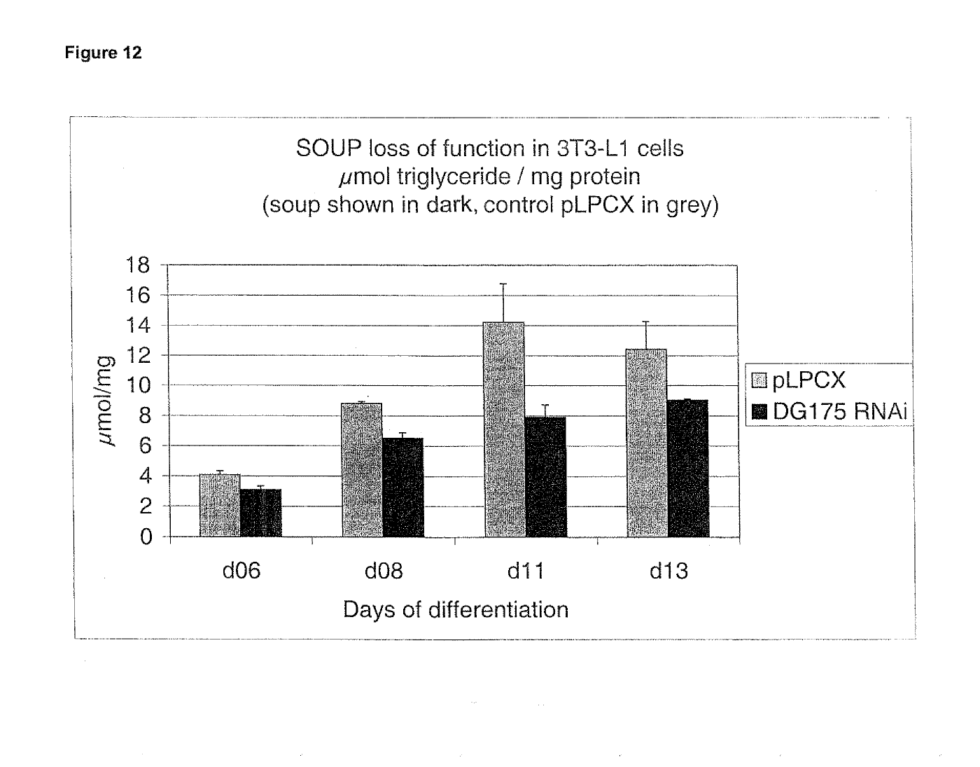

The results are shown in FIG. 12.

Transfection of 3T3-L1 cells (differentiated adipocytes) with the RNAi construct resulted in a suppression of the SOUP1 transcript of about 50%. This suppression resulted in a significantly decreased triglyceride content in the cells (expressed as μmol triglyceride/mg protein) compared to control cells transfected with a control vector (pLPCX). This demonstrates that the suppression of SOUP leads to an increased energy consumption expressed as a decreased content of triglycerides.

In contrast thereto, overexpression of SOUP leads to a phenotype showing an increased triglyceride content (data not shown).

The above result shows that the modulation of the expression of SOUP has a measurable effect on the metabolism of mammalian cells. This proves that SOUP modulators are suitable for the treatment of metabolic diseases.

1. A method for the treatment of a metabolic disorder comprising administering a subject in need thereof, a therapeutically effective amount of a modulator of a SOUP1 polypeptide.

2. The method of claim 1 wherein the subject is a mammal.

3. The method of claim 1, wherein the subject is a human.

4. The method of claim 1, wherein the SOUP1 polypeptide is encoded by the nucleic acid molecule of SEQ ID NOs: 9, 11, 13 or 51 or a nucleic acid molecule which hybridizes at 65° C. in a solution containing 0.2×SSC and 0.1% SDS to a nucleic acid molecule as depicted in SEQ ID NO: 9, SEQ ID NO: 11, SEQ ID NO: 13 and/or SEQ ID NO: 51 and/or the complementary strand thereof.

5. The method of claim 1, wherein the SOUP1 polypeptide is a polypeptide which is at least 35%, preferably at least 50%, more preferably at least 60%, more preferably at least 70%, more preferably at least 80%, more preferably at least 90%, most preferably at least 95% and most preferably at least 99% identical to the amino acid sequence as depicted in any one of SEQ ID NOs: 10, 12, 14 or 52.

6. The method of claim 1, wherein the metabolic disorder is selected from obesity, adipositas, eating disorders (bulimia nervosa, anorexia nervosa), cachexia (wasting), pancreatic dysfunction and/or a disorder related to ROS production.

7. The method of claim 1, wherein the modulator is selected from an antibody, fragment or derivative thereof, an aptamer, or an anti-sense oligonucleotide.

8. The method of claim 1, wherein the modulator is a SOUP1 agonist.

9. The method of claim 1, wherein the modulator is a SOUP1 antagonist.
